(19) United States
(12) Patent Application Publication
(10) Pub. No.: US 2021/0252265 A1
(43) Pub. Date: Aug. 19, 2021

(54) DEVICES AND METHODS FOR ULTRASONIC DELIVERY OF AN AGENT WITHIN AN ORAL CAVITY

(71) Applicant: Suono Bio, Inc., Cambridge, MA (US)

(72) Inventors: Carl Schoellhammer, Cambridge, MA (US); Norman F. Sheppard, Cambridge, MA (US); Marion France, Cambridge, MA (US)

(73) Assignees: Suono Bio, Inc., Cambridge, MA (US); Suono Bio, Inc., Cambridge, MA (US)

(21) Appl. No.: 17/259,223

(22) PCT Filed: Jul. 19, 2019
(86) PCT No.: PCT/US2019/042519

§ 371 Date: Jan. 11, 2021

Related U.S. Application Data
(60) Provisional application No. 62/791,277, filed on Jan. 11, 2019.
(60) Provisional application No. 62/701,408, filed on Jul. 20, 2018.

(51) Int. Cl. A61M 37/00

(52) U.S. Cl. CPC A61M 37/0092; A61M 2037/0007; A61M 2205/3368; A61M 2205/587; A61M 2210/0625

(57) ABSTRACT

The invention provides devices and methods for delivery of an agent to buccal tissue of a subject. The devices and methods use transient acoustic cavitation to transfer an agent directly from a fluid to buccal tissue.

### CROSS-REFERENCE TO RELATED APPLICATIONS

This application claims the benefit of, and priority to, U.S. Provisional Patent Application No. 62/791,277, filed Jan. 11, 2019, and U.S. Provisional Patent Application No. 62/701,408, filed Jul. 20, 2018, the contents of each of which are incorporated by reference.

### FIELD OF THE INVENTION

The invention relates generally to devices and methods for ultrasonic delivery of an agent within an oral cavity.

### BACKGROUND

Millions of people suffer from diseases for which no effective therapy exists. In many such cases, the molecular basis of the disease is known, but current reagents and delivery mechanisms are ineffective at modifying the activity of one or more molecular targets that cause the condition. So-called “undruggable” targets are associated with a wide array of serious diseases, including cancers, inflammatory diseases, and gastrointestinal disorders.

Delivery of a drug through the gastrointestinal (GI) tract is desirable because it can be performed rapidly and with minimal invasion of the patient's body. However, GI delivery does not work for many drugs. For example, biological therapeutics (“biologics”), which generally consist of large macromolecules, are poorly absorbed through the GI tract. Many organic small molecule drugs have low bioavailability when provided orally due to first-pass metabolism, a process by which enzymes of the gut and liver modify chemical compounds before they enter systemic circulation. Absorption may also be limited if the patient has a diarrhea, which minimizes the duration of transit of the drug through the GI tract.

Many biologics are administered intravenously, but this mode of administration has its own set of obstacles. For example, circulating biologics trigger the body's immune response, which results in destruction of the drug or its elimination from the body and nullifies its therapeutic benefit. Therefore, biologics are typically formulated in encapsulated structures or macromolecular complexes that allow them to evade detection by the immune system.

Consequently, in many instances, advances in the understanding of the biological mechanism of a disease have not been translated into effective therapies, and people continue to suffer from conditions that cannot be adequately treated.

### SUMMARY

The invention provides devices and methods for ultrasonic delivery of agents into buccal tissue of a patient. The devices promote transient acoustic cavitation of fluid in contact with buccal tissue to promote transfer of agents from the fluid into the tissue. Transfer occurs rapidly, often within a minute or less, which minimizes exposure of the agent to both sonic energy and physiological stresses. The devices permit transfer of a broad range of agents, including organic small molecules and biological macromolecules. Because the agent is not ingested, the effects of first-pass metabolism are avoided. Moreover, use of the devices obviates the need to provide a drug, e.g., a biologic drug, in a protective formulation, such as an encapsulated structure or macromolecular complex. Consequently, the devices allow direct administration of pharmacological agents, such as biologics (e.g., nucleic acids such as siRNAs, mRNAs), and organic small molecules, in unmodified (i.e., native) forms that preserve their activity.

By providing improved delivery of therapeutic agents to buccal tissue, the devices and methods unlock the therapeutic potential of a variety of agents. For certain agents that must be provided at high doses to achieve a therapeutic benefit with prior methods, methods of the invention achieve comparable therapeutic effect using greatly reduced dosages and/or less frequent administration. In other cases, the devices and methods allow therapeutically effective delivery of agents that previously had no clinically useful formulation or delivery mechanism. As a result, the invention allows for pharmaceutical intervention for many molecular targets that were previously considered “undruggable” and provides effective treatments for a multitude of diseases and disorders.

Administration of agents using the devices and methods of the invention also offers superior convenience. Ultrasonic delivery of agents via the GI tract has been described previously, but prior methods require positioning of an ultrasound device in the colon. In prior methods, the device must be inserted through the rectum or allowed to pass through the GI tract after being swallowed. However, because the devices of the invention deliver a drug to the inside of the cheek, they can be placed in the mouth. Additionally, because transfer is rapid, the entire procedure is quick and requires minimal patient preparation.

In an aspect, the invention provides devices for delivering an agent into buccal tissue of a subject. The devices include an ultrasound transducer, a fluid chamber, and an ultrasound horn. The fluid chamber fits within an oral cavity of a subject, is fluidically sealed from the ultrasound transducer, and has an opening. The ultrasound horn is operably coupled to the ultrasound transducer and extends into the fluid chamber.

The devices may include an enclosure surrounding a portion of the fluid chamber. The enclosure may surround all of the fluid chamber but the opening. The enclosure may contain a membrane that is permeable to gas but impermeable to liquid. The enclosure may contain one or more exhaust channels. The enclosure may contain a port that fluidically connects the fluid chamber to a fluid source. The enclosure may dampen the sound of the ultrasound horn.

The devices may include an illumination source that illuminates the opening of the fluid chamber. The illumination source may be a light, light-emitting diode, or laser.

The devices may include a thermocouple that operably couples the fluid chamber to the ultrasound transducer.

The devices may include a circuit breaker that is operably coupled to the ultrasound transducer. The circuit breaker may terminate a signal to the ultrasound transducer in response to a stimulus. The stimulus may be a time, temperature, resistivity, or voltage.

The device may include a control unit operably coupled to the ultrasound transducer. The control unit may contain one or more of an input mechanism, an output mechanism, a logic board, and an ultrasound driver board.

The device may include a fluid source. The device may be coupled to a fluid source. The fluid source may be fluidically connected to the fluid chamber.

The device may include a disposable tip. The tip may contain the fluid chamber.

In an aspect, the invention provides methods for delivering an agent into buccal tissue of a subject. The methods include introducing a chamber containing a fluid and an agent into an oral cavity of a subject and delivering ultrasound energy to the fluid in the chamber at a frequency to produce bubbles within the fluid and cause transient cavitation of the bubbles. Implosion of the bubbles propels the agent in the fluid from the chamber and into the buccal tissue of the subject.

In aspect, the invention provides methods for delivering a nucleic acid into buccal tissue of a subject. The methods include introducing a chamber containing a fluid and a nucleic acid into an oral cavity of a subject, and delivering ultrasound energy to the fluid in the chamber to thereby cause the nucleic acid in the fluid to exit the chamber and enter the buccal tissue of the subject. The methods may include introducing the agent into the chamber.

The ultrasound energy may be delivered at a frequency of from about 10 kHz to about 10 MHz. Preferably, the ultrasound energy is delivered at a frequency of less than 100 kHz. For example, the ultrasound energy may be delivered at a frequency of from about 20 kHz to about 60 kHz. The ultrasound energy may be delivered at a frequency of about 40 kHz.

The ultrasound energy may be delivered in a pulse. The pulse may be less than 20 minutes, less than 10 minutes, less than 5 minutes, or less than 2 minutes. The pulse may be from about 0.1 seconds to about 3 minutes. The pulse may be about 10 minutes, about 5 minutes, about 3 minutes, about 2 minutes, about 1 minute, about 30 seconds, about 20 seconds, or about 10 seconds. The pulse may include a duty cycle in which the ultrasound energy is applied intermittently or with gaps within the pulse. For example, the pulse may include two or more “on” periods separated by “off” periods. The “on” and “off” periods may be of any duration. For example and without limitation, the “on” and/or “off” periods may be about 10 milliseconds, about 20 milliseconds, about 50 milliseconds, about 0.1 seconds, about 0.2 seconds, about 0.5 seconds, about 1 second, about 2 seconds, about 5 seconds, about 10 seconds, about 20 seconds, about 30 seconds, about 1 minute, about 2 minutes or about 5 minutes. The pulse may have a duty cycle that includes any ratio of “on” and “off” periods. For example, the pulse may have a 10%, 20%, 30%, 40%, 50%, 60%, 70%, 80%, 90%, or 100% duty cycle.

The ultrasound energy may be selected so that it does not result in breakdown of a fraction or percentage of the agent. For example, the ultrasound energy may result in breakdown of less than about 95% of the agent, less than about 90% of the agent, less than about 80% of the agent, less than about 70% of the agent, less than about 60% of the agent, less than about 50% of the agent, less than about 40% of the agent, less than about 25% of the agent, or less than about 10% of the agent.

The pulse may have an intensity of about 0.1 W/cm², about 0.2 W/cm², about 0.5 W/cm², about 1 W/cm², about 2 W/cm², about 5 W/cm², about 10 W/cm², about 20 W/cm², about 50 W/cm², or about 100 W/cm².

The agent may be any agent that provides a therapeutic benefit. For example, the agent may be a nucleic acid, a peptide, a polypeptide, a protein, an antibody, an organic molecule, or any combination thereof. The agent may be unformulated, i.e., it may exist substantially as a free molecule (e.g., native form). The agent may not be encapsulated. For example, the agent may not be contained in a viral particle, a viral capsid, a liposome, a vesicle, or a micelle. The agent may not be in a complex with other macromolecules. For example, the agent may not be in a protein heterocomplex, protein homocomplex, ribonucleoprotein particle, nucleoprotein particle, or nucleic acid multimer. The agent may be a naked nucleic acid. The agent may be a component of a gene editing system, such as the CRISPR system.

The nucleic acid may be RNA, DNA, hybrid RNA-DNA, or a modified nucleic acid, i.e., a nucleic acid that contains non-naturally-occurring nucleotides, such as nucleotides joined by phosphorothioate linkages. The nucleic acid may encode a polypeptide. For example, the nucleic acid may be mRNA or cDNA. The nucleic acid may inhibit, promote, or alterexpression of a gene or polypeptide. For example, the nucleic acid may be siRNA or miRNA.

The methods may result in transfer of at least a minimum amount of the agent from the chamber to the buccal tissue. For example, the method may result in transfer of at least 1% of the agent, at least 2% of the agent, at least 5% of the agent, at least 10% of the agent, at least 20% of the agent, at least 30% of the agent, or at least 40% of the agent.

The fluid may be a liquid, such as an aqueous liquid. The liquid may have a viscosity that does not exceed a certain value. The liquid may have a dynamic viscosity that does not exceed a certain value. For example, the liquid may have a dynamic viscosity that is not greater than about 0.001 mPa·s, not greater than about 0.002 mPa·s, not greater than about 0.005 mPa·s, not greater than about 0.01 mPa·s, not greater than about 0.02 mPa·s, not greater than about 0.05 mPa·s, not greater than about 0.05 mPa·s, not greater than about 0.25 mPa·s, not greater than about 0.5 mPa·s, not greater than about 0.75 mPa·s, not greater than about 1 mPa·s, not greater than about 1.25 mPa·s, not greater than about 1.5 mPa·s, not greater than about 2 mPa·s, not greater than about 3 mPa·s, not greater than about 4 mPa·s, not greater than about 5 mPa·s, not greater than about 10 mPa·s, not greater than about 20 mPa·s, not greater than about 50 mPa·s, or not greater than about 100 mPa·s. The liquid may have a kinematic viscosity that does not exceed a certain value. For example, the liquid may have a kinematic viscosity that is not greater than about 0.001 cSt, not greater than about 0.002 cSt, not greater than about 0.005 cSt, not greater than about 0.01 cSt, not greater than about 0.02 cSt, not greater than about 0.05 cSt, not greater than about 0.1 cSt, not greater than about 0.25 cSt, not greater than about 0.5 cSt, not greater than about 0.75 cSt, not greater than about 1 cSt, not greater than about 1.25 cSt, not greater than about 1.5 cSt, not greater than about 2 cSt, not greater than about 3 cSt, not greater than about 4 cSt, not greater than about 5 cSt, not greater than about 10 cSt, not greater than about 20 cSt, not greater than about 50 cSt, or not greater than about 100 cSt.

The fluid may contain the agent or nucleic acid. The agent or nucleic acid may be dissolved in the fluid. The agent or nucleic acid may be suspended in the fluid.

The fluid may contain an excipient. The excipient may facilitate transfer of the agent. The excipient may facilitate analysis or quantification of transfer of the agent. The excipient may be 1,2,4,5 benzenetetracarboxylic acid, 3,3′ thiodipropione acid, 8-arm poly(ethylene glycol), adipic acid, alpha-cyclodextrin, cysteine, didodecyl 3,3′-thiodipropionate, EDTA, fructose, glycerin, mannose, mucin, poloxamer 407, poly(lactide glycolide) acid, poly(vinyl alcohol), polyethoxylated castor oil, saccharin, sodium glycolate, sodium glycocholate, sodium taurodeoxycholate, or sodium thiosulfate.

The methods may include repeating one or more of the steps. For example, the steps may be performed two, three, four, five, or more times. The repeated steps may be interspaced by a time interval, such as 5 minutes, 10 minutes, 15 minutes, 30 minutes, 1 hour, 2 hours, 4 hours, 4 hours, 1 day, 2 days, 3 days, 4 days, 5 days, 1 week, 2 weeks, 3 weeks, 4 weeks or more.

The methods may include repeated a method described above according to a schedule. The schedule may include repeated administrations of an agent to buccal tissue at defined intervals for a defined period. For example, schedule may include repeated administration at intervals of about 1 day, about 2 days, about 3 days, about 4 days, about 5 days, or about 7 days over a period of about 1 week, about 2 weeks, about 3 weeks, about 4 weeks, about 6 weeks, about 8 weeks, or about 12 weeks.

The subject may be any type of subject, such as an animal, for example, a mammal, for example, a human. The subject may suffer from a disease, disorder or condition for which delivery of an agent to buccal tissue can provide a benefit. The disease, disorder, or condition may be or include amalgam tattoo, angular cheilitis, aphthous stomatitis, Bednar's aphthae, Behcet's disease, burning mouth syndrome, cancer, candidiasis, canker sores, cold sores, Crohn's disease, eosinophilic esophagitis, foot-and-mouth disease, gastrointestinal disorders, geographic tongue, gingivitis, gum disease, herpangina, herpes labialis, herpes simplex, infection, inflammatory bowel disease, inflammatory diseases, leukoplakia, lichen planus, mucositis, oral mucositis, pemphigus vulgaris, periodontitis, pharyngitis, side effect of chemotherapy, side effect of radiation therapy, Sjogren's syndrome, Sutton's disease, temporomandibular joint syndrome, transient lingual papillitis, ulcers, ulcerative colitis, or xerostomia.

The methods may include the use of a device of the invention, such as one of the devices described above in relation to devices of the invention.

In an aspect, the invention provides methods for delivering an agent into buccal tissue of a subject. The methods include providing a device that includes an ultrasound transducer; a fluid chamber that is configured to fit within an oral cavity of a subject, is fluidically sealed from the ultrasound transducer, and has an opening; and an ultrasound horn that is operably coupled to the ultrasound transducer and extends into the fluid chamber. The methods include filling the fluid chamber with a fluid that contains an agent; introducing the device to the oral cavity of a subject such that the opening of the device contacts buccal tissue; and delivering, via the device, ultrasound energy to the fluid to thereby cause the agent in the fluid to exit the fluid chamber and enter the buccal tissue of the subject. The device may include any of the elements described above in relation to devices of the invention. The methods may include any of the elements described above in relation to other methods of the invention.

### BRIEF DESCRIPTION OF THE DRAWINGS

FIG. 1 shows an ultrasound device according to an embodiment of the invention.

FIG. 2 shows an ultrasound device according to an embodiment of the invention.

FIG. 3 shows an ultrasound device according to an embodiment of the invention.

FIG. 4A is perspective view of a device according to an embodiment of the invention.

FIG. 4B is a top view of the device shown in FIG. 4A.

FIG. 4C is a bottom view of the device shown in FIG. 4A.

FIG. 4D is a left side view of the device shown in FIG. 4A.

FIG. 4E is a right side view of the device shown in FIG. 4A.

FIG. 4F is a front view of the device shown in FIG. 4A.

FIG. 4G is a rear view of the device shown in FIG. 4A.

FIG. 4H is a section view of the device shown in FIG. 4A.

FIG. 5 is a shaded perspective view of a tip of a device according to an embodiment of the invention.

FIG. 6 is a lined perspective view of a tip of a device according to an embodiment of the invention.

FIG. 7 is a sectioned perspective section view of a tip of a device according to an embodiment of the invention.

FIG. 8 is a shaded bottom view of a tip of a device according to an embodiment of the invention.

FIG. 9 is a lined bottom view of a tip of a device according to an embodiment of the invention.

FIG. 10 is a schematic of the procedure used to test delivery of budesonide to treat or prevent ulcers induced by acetic acid in hamster cheek pouches.

FIG. 11 is a graph of ulcer area at various time points after administration of acetic acid to hamster cheek pouches.

FIG. 12 shows graphs of necrotic area at various time points after administration of acetic acid to hamster cheek pouches.

FIG. 13 shows histopathological images of hamster cheek pouches after administration of acetic acid and graph of microscopic scores of the histopathological images.

FIG. 14 shows graphs of ulcer area at various time points after administration of acetic acid to hamster cheek pouches.

FIG. 15 is a graphic abstract of the ultrasound devices and methods for treatment of oral lesions according to an embodiment of the invention.

FIG. 16 is an illustration of the oral ultrasound device for local administration of therapeutics to mucosal surfaces.

FIG. 17 shows a 3D acoustic pressure map of the horn in FIG. 16.

FIG. 18 is a schematic demonstrating delivery of fluorescently-labeled dextrans to small intestinal porcine biopsy punches using the oral ultrasound device.

FIG. 19 shows representative fluorescent images of porcine mucosal tissue after delivery of dextrans of various molecular weights.

FIG. 20 is a graph of fluorescence intensity of the porcine mucosa measured immediately after delivery of dextrans.

FIG. 21 is a graph of the temperature rise during ultrasound treatment on porcine small intestine.

FIG. 22 shows representative images of the oral mucosa of dogs before and after ultrasound treatment.

FIG. 23 is a schematic of the three experimental groups and respective disease induction and treatment regimen using ultrasound-mediated budesonide delivery to treat oral lesions in hamster cheek pouches in vivo.

FIG. 24 shows representative images of oral lesions in the disease group (untreated) over an 8-day period.

FIG. 25 is a graph showing that oral lesion areas remain smaller in the budesonide+US treatment group following 24 and 48 h following lesion induction with acetic acid.

FIG. 26 shows representative images of macroscopic oral lesion areas at 48 h post acetic acid application.

### DETAILED DESCRIPTION

In modern biomedicine, identification of a pharmacological treatment for a disease typically occurs in the following sequence of steps: understanding of the molecular basis of the disease, identification of an agent that counteracts the aberrant molecular phenomenon, and development of a pharmaceutical composition or methodology that allows delivery of the agent to the appropriate tissue or location in the body to combat the disease. Each phase requires a substantial investment of time, human effort, and financial resources. However, delays during the third phase are particularly frustrating due to the sense that the solution is nearly in hand while people continue to suffer from the disease.

Myriad barriers can block or delay development of an agent that alters the activity of a disease-causing target in vitro into a useful therapeutic. For example, when organic small molecules are administered orally, they may be modified by enzymes of the gut and liver during first-pass metabolism before they enter circulation. When this occurs, only a small percentage of the administered dose of the compound is available to affect its biological target. Many hydrophilic small molecules are unable to pass through cell membranes and thus must be formulated to facilitate cellular uptake. Conversely, hydrophobic small molecules often have poor solubility, a feature that must be overcome to allow such compounds to be distributed throughout the body via the circulatory system.

A distinct but overlapping set of problems faces molecules of biological origin. For example, inhibitory RNAs, such as siRNAs and miRNAs, hold great therapeutic promise due to the ease with which they can be adapted to affect different targets, but several technical obstacles must be overcome to make effective pharmacological agents from them. One problem is the susceptibility of these small RNA molecules to degradation by enzymes in serum and tissues. Another issue is that siRNAs exert their effects inside cells but do not enter cells readily. Consequently, siRNA-based therapeutics typically contain siRNA molecules that are encapsulated or complexed with other macromolecules to promote cellular uptake. A broader problem that confronts not only interfering RNAs but all biological therapeutics, such as proteins, antibodies, and nucleic acids for gene therapy, is their immunogenicity. When foreign macromolecules enter circulation, they are recognized by the immune system as foreign and destroyed and/or eliminated. Thus, to retain their efficacy, biologics must be modified in a way that masks their immunogenic elements without interfering with their biological function.

The invention overcomes the aforementioned issues by providing devices and methods for direct delivery of an active pharmacological agent to buccal tissue of a subject. The devices include a fluid chamber that contains an ultrasound horn and an opening that contacts buccal tissue. When the agent is loaded into the fluid chamber, a signal from the ultrasound horn causes transient acoustic cavitation of fluid in the chamber that transfers the agent into the buccal tissue. Because the transfer occurs directly from the fluid chamber to buccal tissue, the agent need not be provided in a special formulation, such as an encapsulated format or macromolecular complex with other molecules that are not pharmacologically active. In addition, the use of transient cavitation allows the transfer to occur rapidly, typically within minutes. One benefit of the rapid transfer is that exposure of the agent to ultrasonic vibration, which can cause unfolding or breakdown, is minimized and the activity of the agent is preserved. The rapid transfer also makes the procedure easier and less burdensome for patients and physicians.

### Ultrasound Devices

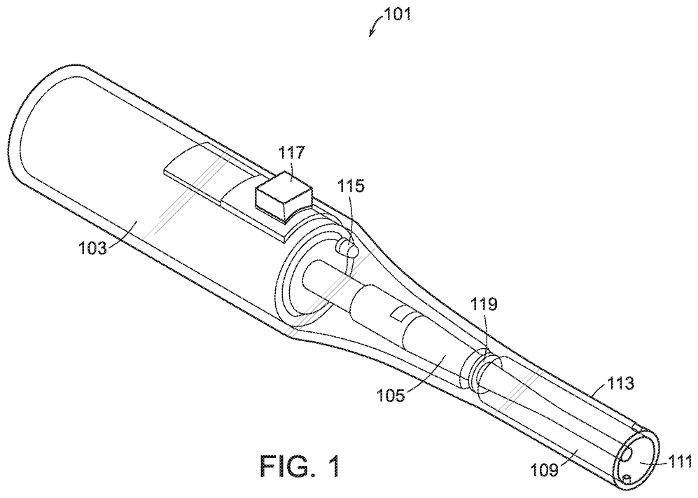

FIG. 1 shows an ultrasound device 101 according to an embodiment of the invention. The device 101 includes an ultrasound transducer 103 coupled to an ultrasound horn 105. The ultrasound horn 105 extends into a fluid chamber 109 that is fluidically separated from the ultrasound transducer 103. The fluid chamber 109 includes at least one opening 111 that can be positioned against buccal tissue when the tip of the device 101 is inserted into the oral cavity of a subject. Suitable ultrasound transducers 103 include those sold under the trade names VCX 500 and VCX 130 (Sonics & Materials, Inc.; Newtown, Conn.). The ultrasound horn may be half-wave horn or a full-wave horn. Suitable ultrasound transducers 103 and ultrasound horns 105 are described in, for example, Schoellhammer, C. M., Schroeder, A., Maa, R., Lauwers, G. Y., Swiston, A., Zervas, M., et al. (2015) Ultrasound-mediated gastrointestinal drug delivery, Science Translational Medicine, 7(310), 310ra168-310ra168, doi: 10.1126/scitranslmed.aaa5937; Schoellhammer, C. M & Traverso, G., Low-frequency ultrasound for drug delivery in the gastrointestinal tract. Expert Opinion on Drug Delivery, 2016, doi: 10.1517/17425247.2016.1171841; Schoellhammer C. M., et al., Ultrasound-mediated delivery of RNA to colonic mucosa of live mice. Gastroenterology, 2017, doi: 10.1053/j.gastro.2017.01.002; and U.S. Publication Nos. 2014/0228715 and 2018/0055991, the contents of each of which are incorporated herein by reference. The fluid chamber may have a volume of about 0.1 ml, about 0.2 ml, about 0.5 ml, about 1 ml, about 2 ml, about 5 ml, or about 10 ml.

The device 101 may include an enclosure 113 that surrounds a portion of the fluid chamber 109. The enclosure 113 may also surround a portion of the ultrasound transducer 103, as shown. The enclosure 113 may dampen sound produced by the device 101. For example, the enclosure 113 may inhibit transmission of sound waves in directions other than toward the opening 111 of the fluid chamber 109. To provide reproducible ultrasound exposure, the distance between the distal tip of the ultrasound horn 105 and the opening 111 of the enclosure may be fixed. For example, the distance between the distal tip and the opening 111 may be about 1 cm, about 2 cm, about 3 cm, about 4 cm, about 5 cm, about 6 cm, or about 8 cm.

The device 101 may include an illumination source 115 that illuminates the opening 111 of the fluid chamber 109. The illumination source 115 may be any type that facilitates positioning of the opening 111 of the device 101 against buccal tissue of the subject. The illumination source 115 may be a light, such as an incandescent light, light-emitting diode, or laser. Other illumination sources 115 are known in the art and described in, for example, . . . .

The device 101 may include an actuator 117 that activates the ultrasound transducer 103. The actuator 117 may be a binary on/off switch, or it may have a range of power settings for the ultrasound transducer 103.

The device 101 may include a separator 119 that separates the fluid chamber 109 from the section of the device 101 that houses the ultrasound transducer 103. The separator may be a gasket or O-ring, as shown. In embodiments in which the ultrasound horn 105 extends linearly into the fluid chamber, as shown, the separator 119 should be positioned at a node of vibration of the ultrasound horn 105.

The fluid chamber 109 may be contained in a tip of the device 101, such as a disposable tip. For example, the tip may comprise a cartridge that is fastened onto the front end of the device 101. The cartridge may contain a film or protective that is punctured by the ultrasound horn 105 when the cartridge is placed on the front of the device, thereby allowing the ultrasound horn 105 to contact the liquid in the fluid chamber 109. Such an arrangement allows the device 101 to be used repeatedly merely by replacing the cartridge at the tip and also facilitates preparation and storage of the liquid and agent within the fluid chamber.

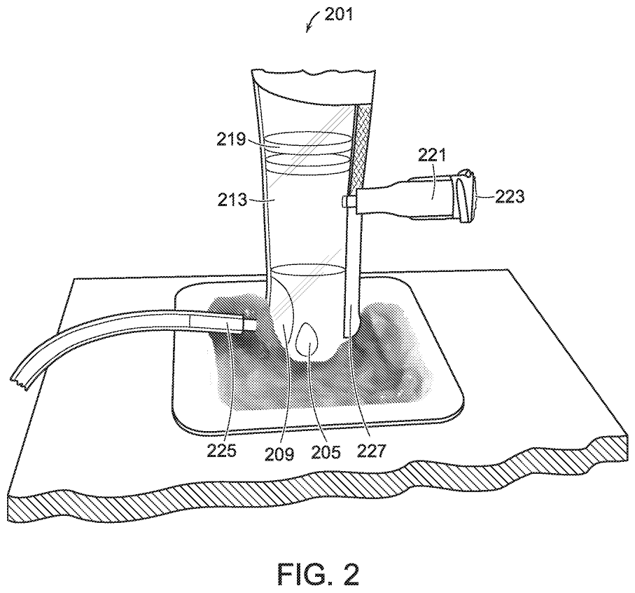

FIG. 2 shows an ultrasound device 201 according to an embodiment of the invention. The device 201 includes an ultrasound horn 205 that extends into a fluid chamber 209. As described above, the device 201 may include an enclosure 213 that surrounds a portion of the fluid chamber 209 and/or a separator 219 that separates the fluid chamber 209 from the ultrasound transducer.

The enclosure 213 may contain a vent 221 to allow the exchange of gas between the fluid chamber 209 and the ambient air. The vent 221 may include exhaust microchannels. Exhaust microchannels facilitate filling the fluid chamber with liquid by permitting release of gas from the chamber. The vent 221 with exhaust microchannels may be positioned on the enclosure at any point that allows upward release of gas when the device is oriented to deliver the agent to the subject, thus avoiding the need to hold the device with the tip downward during use. Additionally or alternatively, the vent 221 may contain a membrane 223 that is permeable to gas but impermeable to liquid. Many gas-permeable, liquid-impermeable materials are known in the art and described in, for example, U.S. Pat. Nos. 3,953,566; 4,152,482; 4,391,873; 4,500,328; 4,520,056; 4,772,508; 4,957,522; 5,522,769; and 6,676,871; and U.S. Publication No. 2010/0107878. For example and without limitation, the membrane may contain one or more of ethyl cellulose, ethyl/vinyl acetate, ethylene/acrylic acid copolymers, ethylene/alpha-olefin copolymers, ethylene/ethyl acrylate and, ethylene/methyl acrylate, fluoropolymers, fluorosilicone derived from, fluorovinylmethylsilicone, homopolymer polyethylenes, metallocene polypropylenes, nitrate butadiene rubber (NBR), nitrile rubber, poly(4-methyl-1-pentene), polydimethylsiloxane, polydimethylsiloxane, polyethylene, polyimides, polyisoprene, polyoctenamer, polyolefin, polyphenylvinylmethylsiloxane, polypropylene materials, polypropylene, polyethylene, polypropylenes, polytetrafluoroethylene, polyurethanes, polyvinylmethylsiloxane, propylene/alpha-olefin copolymers, propylene/ethylene copolymer, radical low-density polyethylenes, tetrafluoroethylene-(perfluoroalkyl) vinyl ether copolymer (PFA), and tetrafluoroethylene-hexafluoropropylene copolymer (FEP).

The enclosure 213 may contain a port 225 that fluidically connects the fluid chamber to a fluid source. The fluid source may be a component of the device 201 or may be functionally coupled to the device 201.

The device 201 may contain one or more safety features that prevent events that could be harmful to the subject, such as excessive heating of the fluid or transmission of electrical signal. For example, the device 201 may contain a thermocouple 227 that couples the fluid chamber to the ultrasound transducer. The thermocouple 227 can provide negative feedback to inactivate that ultrasound transducer when the temperature of the fluid and/or tissue becomes elevated beyond a threshold value. The device 201 may contain a circuit breaker that is coupled to the ultrasound transducer and terminates a signal to the ultrasound transducer in response to a stimulus. For example and without limitation, the circuit breaker may terminate the signal to the ultrasound transducer after a certain period of time, when the temperature of the fluid and/or tissue becomes elevated beyond a threshold value, or when the electrical circuit containing the ultrasound transducer reaches a threshold value of resistivity or voltage.

The device 201 may include, or be operably connected to, a control unit. The control unit may include one or more of an input mechanism, an output mechanism, a logic board, and an ultrasound driver board. For example and without limitation, the input mechanism may include buttons, switches, a keyboard, or the like. For example and without limitation, the output mechanism may provide a visual, audible, tactile, or vibrational signal.

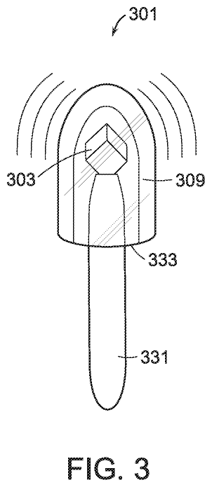

FIG. 3 shows an ultrasound device 301 according to an embodiment of the invention. The device 301 may have generally have a “lollipop” shape that is conducive to delivery of an agent to buccal tissue. Thus, the device 301 may have a handle 331 that can be held by the subject or a person administering the agent to the subject and a tip 333 that can be placed in an oral cavity of the subject. The ultrasonic transducer 303 is contained within a central portion of the tip 333, and the fluid chamber 309 comprises an exterior portion of the tip 333. An opening may be positioned at any point on an exterior surface of the fluid chamber 309 to facilitate contact of the fluid with buccal tissue.

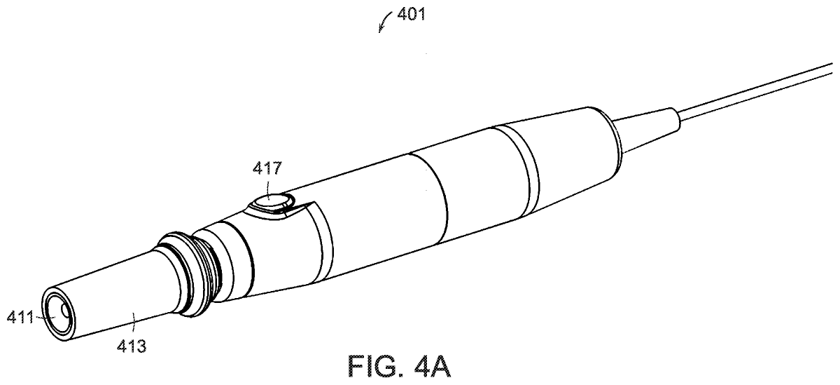

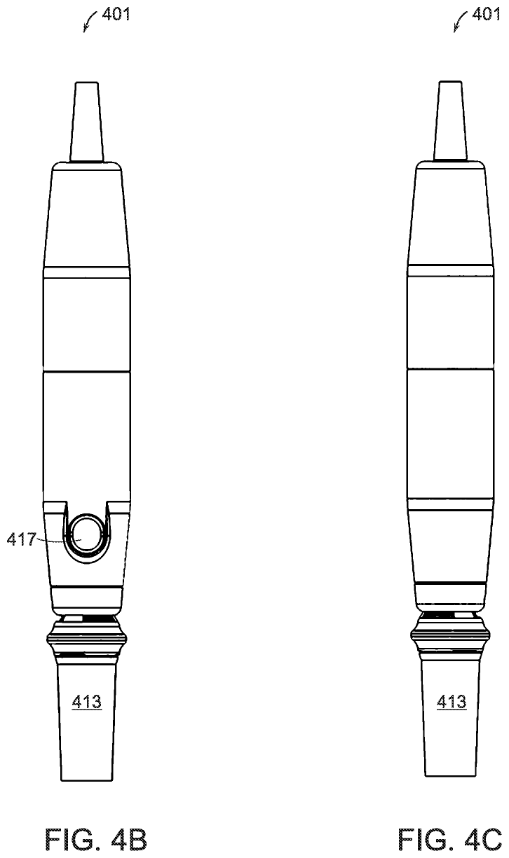

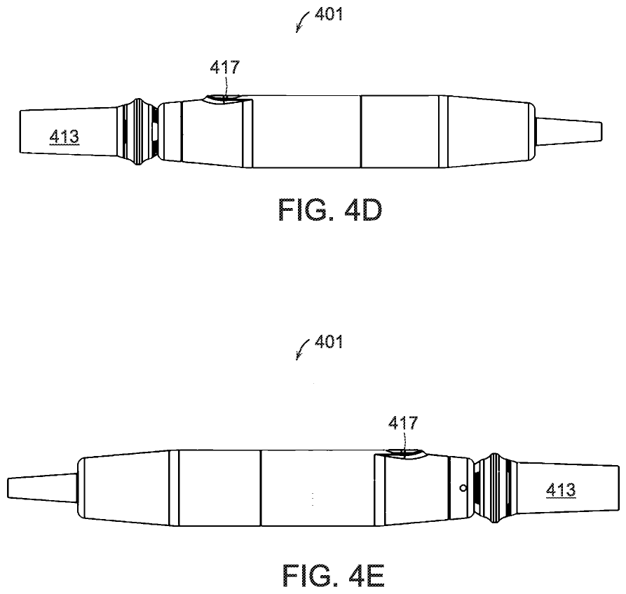

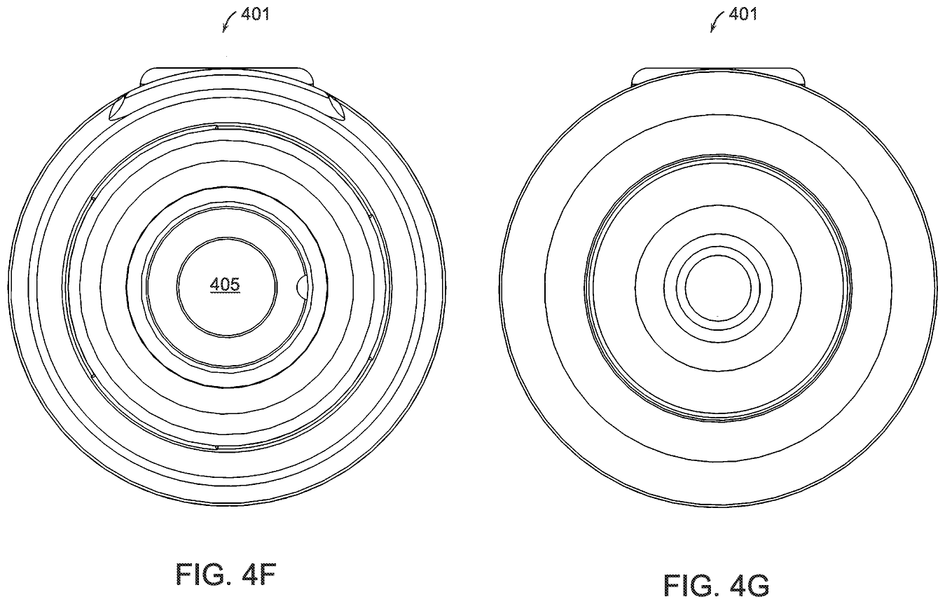

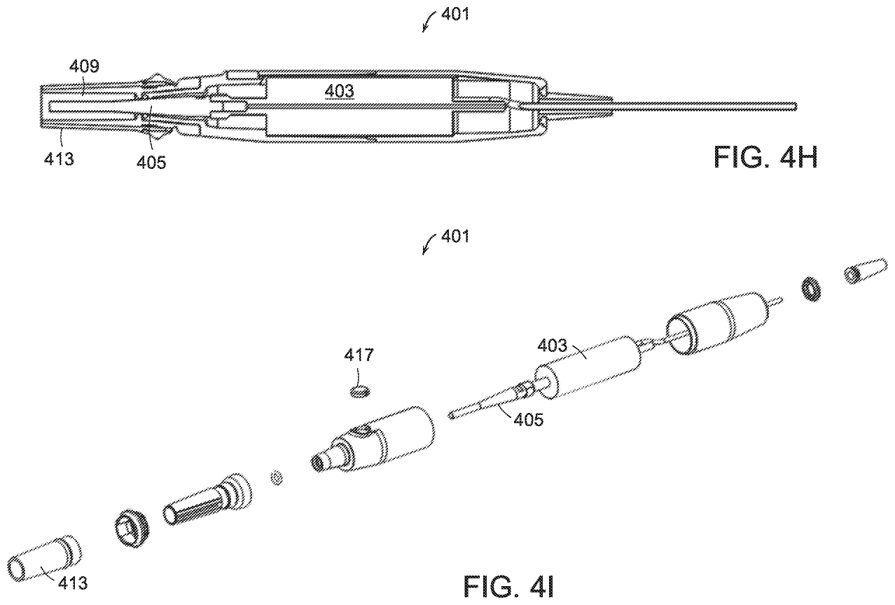

FIG. 4A is perspective view of a device according to an embodiment of the invention. The actuator 417, enclosure 413, and opening 411 are indicated in this view.

FIG. 4B is a top view of the device shown in FIG. 4A. The actuator 417 and enclosure 413 are indicated in this view.

FIG. 4C is a bottom view of the device shown in FIG. 4A. The enclosure 413 is indicated in this view.

FIG. 4D is a left side view of the device shown in FIG. 4A. The actuator 417 and enclosure 413 are indicated in this view.

FIG. 4E is a right side view of the device shown in FIG. 4A. The actuator 417 and enclosure 413 are indicated in this view.

FIG. 4F is a front view of the device shown in FIG. 4A. The ultrasound horn 405 is indicated in this view.

FIG. 4G is a rear view of the device shown in FIG. 4A.

FIG. 4H is a section view of the device shown in FIG. 4A. The enclosure 413, fluid chamber 409, ultrasound horn 405, and ultrasound transducer 403 are indicated in this view.

FIG. 4I is an exploded view of the device shown in FIG. 4A. The enclosure 413, actuator 413, ultrasound horn 405, and ultrasound transducer 403 are indicated in this view.

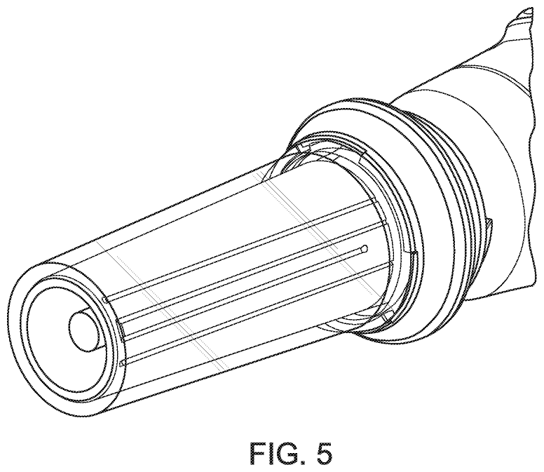

FIG. 5 is a shaded perspective view of a tip of a device according to an embodiment of the invention.

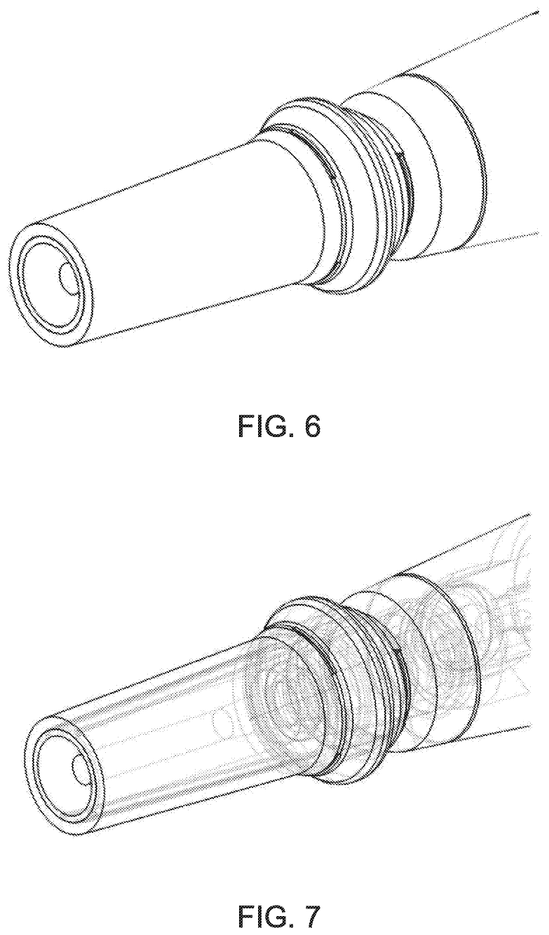

FIG. 6 is a lined perspective view of a tip of a device according to an embodiment of the invention.

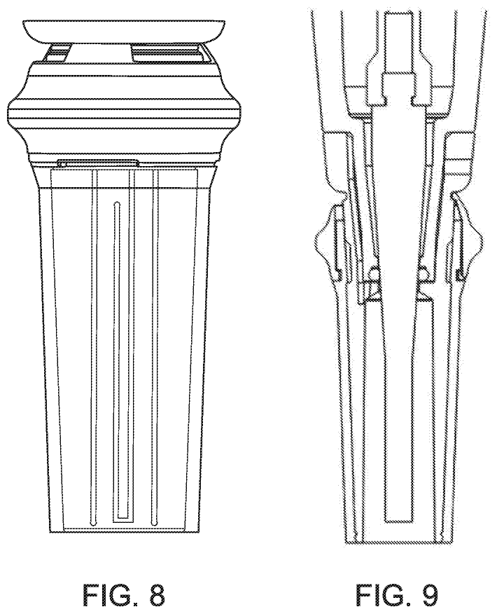

FIG. 7 is a sectioned perspective section view of a tip of a device according to an embodiment of the invention.

FIG. 8 is a shaded bottom view of a tip of a device according to an embodiment of the invention.

FIG. 9 is a lined bottom view of a tip of a device according to an embodiment of the invention.

### Ultrasound Delivery Methods

The invention provides methods of delivering agents to buccal tissue of a subject using devices of the invention. The methods include introducing the fluid chamber of the device into the oral cavity of the subject. The fluid chamber contains fluid and the agent to be delivered. Preferably, the opening of the fluid chamber is in contact with the buccal tissue to be targeted for delivery.

The methods include delivering ultrasound energy to the fluid at a frequency that produces bubbles within the fluid and causes transient cavitation of the bubbles. Gentle implosion of the bubbles produces shock waves that permeabilize cells and propel the agent from the fluid chamber into buccal tissue. Typically, the bubbles have diameters on a micron scale, i.e., from about 1 micron to about 1000 microns. The use of ultrasound to cause transient cavitation to deliver agents to tissue is described in, for example, Schoellhammer, C. M., Schroeder, A., Maa, R., Lauwers, G. Y., Swiston, A., Zervas, M., et al. (2015). Ultrasound-mediated gastrointestinal drug delivery. Science Translational Medicine, 7(310), 310ra168-310ra168, doi: 10.1126/scitranslmed.aaa5937; Schoellhammer, C. M & Traverso, G., Low-frequency ultrasound for drug delivery in the gastrointestinal tract. Expert Opinion on Drug Delivery, 2016, doi: 10.1517/17425247.2016.1171841; Schoellhammer C. M., et al., Ultrasound-mediated delivery of RNA to colonic mucosa of live mice, Gastroenterology, 2017, doi: 10.1053/j.gastro.2017.01.002; and U.S. Publication Nos. 2014/0228715 and 2018/0055991, the contents of each of which are incorporated herein by reference.

The frequency of the ultrasound energy may be between 10 kHz and 10 MHz. Preferably, the frequency of the ultrasound energy is less than less than 100 kHz. For example and without limitation, the frequency may be from about 20 kHz to about 100 kHz, from about 20 kHz to about 80 kHz, from about 20 kHz to about 60 kHz, or from about 30 kHz to about 50 kHz. The frequency may about 20 kHz, about 30 kHz, about 40 kHz, about 50 kHz, or about 60 kHz.

In some embodiments, the ultrasound energy may be delivered as a pulse, i.e., it may be delivered over a brief, finite period in order to minimize damage to the agent being delivered by the ultrasound energy. For example and without limitation, the pulse may be less than 20 minutes, less than 10 minutes, less than 5 minutes, or less than 10 minutes. For example and without limitation, the pulse may be from about 10 seconds to about 3 minutes. The pulse may be about 10 minutes, about 5 minutes, about 3 minutes, about 3 minutes, about 1 minute, about 30 seconds, about 20 seconds, or about 10 seconds. The pulse may include a duty cycle in which the ultrasound energy is applied intermittently or with gaps within the pulse. For example, the pulse may include two or more “on” periods separated by “off” periods. The “on” and “off” periods may be of any duration. For example and without limitation, the “on” and/or “off” periods may be about 10 milliseconds, about 20 milliseconds, about 50 milliseconds, about 0.1 seconds, about 0.2 seconds, about 0.5 seconds, about 1 second, about 2 seconds, about 5 seconds, about 10 seconds, about 20 seconds, about 30 seconds, about 1 minute, about 2 minutes or about 5 minutes. The pulse may have a duty cycle that includes any ratio of “on” and “off” periods. For example, the pulse may have a 10%, 20%, 30%, 40%, 50%, 60%, 70%, 80%, 90%, or 100% duty cycle.

The parameters of the ultrasound pulse, such as the frequency and/or duration, may be selected so that damage to the agent is limited to a certain fraction or percentage of the agent. For example and without limitation, the ultrasound energy may result in breakdown of less than about 95% of the agent, less than about 90% of the agent, less than about 80% of the agent, less than about 70% of the agent, less than about 60% of the agent, less than about 50% of the agent, less than about 40% of the agent, less than about 25% of the agent, or less than about 10% of the agent.

The pulse may have an intensity of about 0.1 W/cm², about 0.2 W/cm², about 0.5 W/cm², about 1 W/cm², about 2 W/cm², about 5 W/cm², about 10 W/cm², about 20 W/cm², about 50 W/cm², or about 100 W/cm².

The parameters of the ultrasound pulse, such as the frequency and/or duration, may be selected so that at least a minimum amount of the agent is transferred from the chamber to the buccal tissue. For example and without limitation, the ultrasound energy may result in transfer of at least 1% of the agent, at least 2% of the agent, at least 5% of the agent, at least 10% of the agent, at least 20% of the agent, at least 30% of the agent, or at least 40% of the agent.

The pulse may achieve a defined pressure at a defined distance from the opening 111 of the enclosure 113. For example, the pulse may be a pressure of about 10 kPa, 20 kPa, 40 kPa, 60 kPa, 80 kPa, 100 kPa, 125 kPa, 150 kPa, 200 kPa, or 250 kPa at a distance of about 0.1 mm, about 0.2 mm, about 0.5 mm, about 1 mm, about 2 mm, about 3.5 mm, about 5 mm, or about 10 mm from the opening 11.

The pulse may achieve an increase in the temperature of the tissue as the pulse is applied. For example and without limitation, the pulse may increase the temperature of the tissue by about 1° C., about 2° C., about 3° C., about 4° C., about 5° C., about 6° C., about 8° C., about 10° C., about 12° C., about 15° C., about 20° C., or about 25° C. The increase may occur after a duration of about 1 s, about 2 s, about 3 s, about 5 s, about 10 s, about 20 s, about 30 s, about 60 s, about 2 minutes, about 5 minutes, about 10 minutes, about 15 minutes, or about 20 minutes.

In preferred embodiments, the fluid is a liquid in which the agent is dissolved, suspended, or otherwise uniformly distributed throughout the fluid. Preferably, the fluid is an aqueous liquid. The aqueous liquid may contain other components that stabilize the agent, such as salts, buffers, osmotic stabilizers, and the like.

The fluid should be a liquid conducive to transient acoustic cavitation. Generally, liquids with higher viscosity have a higher threshold for nucleation of bubbles and thus make transient cavitation more difficult. Consequently, in preferred embodiments, the fluid is a liquid with low viscosity. The liquid may have a viscosity that does not exceed a certain value. The liquid may have a dynamic viscosity that does not exceed a certain value. For example and without limitation, the liquid may have a dynamic viscosity that is not greater than about 0.25 mPa·s, not greater than about 0.5 mPa·s, not greater than about 0.75 mPa·s, not greater than about 1 mPa·s, not greater than about 1.25 mPa·s, or not greater than about 1.5 mPa·s. The liquid may have a kinematic viscosity that does not exceed a certain value. For example and without limitation, the liquid may have a kinematic viscosity that is not greater than about 0.25 cSt, not greater than about 0.5 cSt, not greater than about 0.75 cSt, not greater than about 1 cSt, not greater than about 1.25 cSt, or not greater than about 1.5 cSt.

The agent may be any agent that provides a therapeutic benefit. For example and without limitation, suitable agents include alpha-hydroxy formulations, ace inhibiting agents, analgesics, anesthetic agents, anthelmintics, anti-arrhythmic agents, antithrombotic agents, anti-allergic agents, anti-angiogenic agents, antibacterial agents, antibiotic agents, anticoagulant agents, anticancer agents, antidiabetic agents, anti-emetics, antifungal agents, antihypertension agents, antihypotensive agents, antiinflammatory agents, antimicotic agents, antimigraine agents, anti-obesity agents, antiparkinson agents, antirheumatic agents, antithrombins, antiviral agents, antidepressants, antiepileptics, antihistamines, antimuscarinic agents, antimycobacterial agents, antineoplastic agents, antithyroid agents, anxiolytics, asthma therapies, astringents, beta blocking agents, blood products and substitutes, bronchospamolytic agents, calcium antagonists, cardiovascular agents, cardiac glycosidic agents, carotenoids, cephalosporins, chronic bronchitis therapies, chronic obstructive pulmonary disease therapies, contraceptive agents, corticosteroids, cytostatic agents, cystic-fibrosis therapies, cardiac inotropic agents, contrast media, cough suppressants, diagnostic agents, diuretic agents, dopaminergics, elastase inhibitors, emphysema therapies, enkephalins, fibrinolytic agents, growth hormones, hemostatics, immunological agents, immunosupressants, insulins, interferons, lactation inhibiting agents, lipid-lowering agents, lymphokines, muscle relaxants, neurologic agents, NSAIDS, nutraceuticals, oncology therapies, organ-transplant rejection therapies, parasympathomimetics, parathyroid calcitonin and biphosphonates, prostacyclins, prostaglandins, psycho-pharmaceutical agents, protease inhibitors, magnetic resonance diagnostic imaging agents, radio-pharmaceuticals, reproductive control hormones, respiratory distress syndrome therapies, sedative agents, sex hormones, somatostatins, steroid hormonal agents, stimulants and anoretics, sympathomimetics, thyroid agents, vasodilating agents, vitamins, and xanthines. A more complex list of chemicals and drugs that can be used as agents in embodiments of the invention is provided in the The Merck Index: An Encyclopedia of Chemicals, Drugs, and Biologicals Fifteenth Edition, Maryadele J O'Neil, ed., RSC Publishing, 2015, ISBN-13: 978-1849736701, ISBN-10 1849736707, the contents of which are incorporated herein by reference.

Agents may be of any chemical form. For example, agents may be biological therapeutics, such as nucleic acids, proteins, peptides, polypeptides, antibodies, or other macromolecules. Nucleic acids include RNA, DNA, RNA/DNA hybrids, and nucleic acid derivatives that include non-naturally-occurring nucleotides, modified nucleotides, non-naturally-occurring chemical linkages, and the like. Examples of nucleic acid derivatives and modified nucleotides are described in, for example, International Publication WO 2018/118587, the contents of which are incorporated herein by reference. Nucleic acids may be polypeptide-encoding nucleic acids, such as mRNAs and cDNAs. Nucleic acids may interfere with gene expression. Examples of interfering RNAs (RNAi) include siRNAs and miRNAs. RNAi is known in the art and described in, for example, Kim and Rossi, Biotechniques. 2008 April; 44(5): 613-616, doi: 10.2144/000112792; and Wilson and Doudna, Molecular Mechanisms of RNA Interference, Annual Review of Biophysics 2013 42:1, 217-239, the contents of each of which are incorporated herein by reference. Agents may be organic molecules of non-biological origin. Such drugs are often called small-molecule drugs because they typically have a molecular weight of less than 2000 Daltons, although they may be larger. Agents may be combinations or complexes of one or more biological macromolecules and/or one or more small molecules. For example and without limitation, agents may be nucleic acid complexes, protein complexes, protein-nucleic acid complexes, and the like. Thus, the agent may exist in a multimeric or polymeric form, including homocomplexes and heterocomplexes.

The agent may be unformulated, i.e., it may be provided in a biologically active format that does not contain other molecules that interact with the agent solely to facilitate delivery of the agent. Formulations commonly used for delivery of biologic and small-molecule agents include viral particles, viral capsids, liposomes, vesicles, micelles, and complexes with other macromolecules that are not essential for the biological or biochemical function of the agent. Thus, the agent may be provided in a non-encapsulated form or in a form that is not complexed with other molecules unrelated to the function of the agent. For example, the agent may be a naked nucleic acid.

The agent may be a component of a gene editing system, such as a meganuclease, zinc finger nuclease (ZFN), a transcription activator-like effector-based nuclease (TALEN), or the clustered, regularly-interspersed palindromic repeat (CRISPR) system.

Meganucleases are endodeoxyribonucleases that recognize double-stranded DNA sequences of 12-40 base pairs. They can be engineered to bind to different recognition sequences to create customized nucleases that target particular sequences. Meganucleases exist in archaebacterial, bacteria, phages, fungi, algae, and plants, and meganucleases from any source may be used. Engineering meganucleases to recognize specific sequences is known in the art and described in, for example, Stoddard, Barry L. (2006) “Homing endonuclease structure and function” Quarterly Reviews of Biophysics 38 (1): 49-95 doi:10.1017/S0033583505004063, PMID 16336743; Grizot, S.; Epinat, J. C.; Thomas, S.; Duclert, A.; Rolland, S.; Paques, F.; Duchateau, P. (2009) “Generation of redesigned homing endonucleases comprising DNA-binding domains derived from two different scaffolds” Nucleic Acids Research 38 (6): 2006-18, doi:10.1093/nar/gkp1171. PMC 2847234, PMID 20026587; Epinat, Jean-Charles; Arnould, Sylvain; Chames, Patrick; Rochaix, Pascal; Desfontaines, Dominique; Puzin, Clémence; Patin, Amélie; Zanghellini, Alexandre; Pâques, Frédéric (2003 Jun. 1) “A novel engineered meganuclease induces homologous recombination in yeast and mammalian cells” Nucleic Acids Research 31 (11): 2952-2962; and Seligman, L. M.; Chisholm, K M; Chevalier, B S; Chadsey, M S; Edwards, S T; Savage, J H; Veillet, A L (2002) “Mutations altering the cleavage specificity of a homing endonuclease” Nucleic Acids Research 30 (17): 3870-9, doi:10.1093/nar/gkf495. PMC 137417, PMID 12202772, the contents of each of which are incorporated herein by reference.

ZFNs are are artificial restriction enzymes that have a zinc finger DNA-binding domain fused to a DNA-cleavage domain. ZFNs can also be engineered to target specific DNA sequences. The design and use of ZFNs is known in the art and described in, for example, Carroll, D (2011) “Genome engineering with zinc-finger nucleases” Genetics Society of America 188 (4): 773-782, doi:10.1534/genetics.111.131433. PMC 3176093, PMID 21828278; Cathomen T, Joung J K (July 2008) “Zinc-finger nucleases: the next generation emerges” Mol. Ther. 16 (7): 1200-7, doi:10.1038/mt.2008.114, PMID 18545224; Miller, J. C.; Holmes, M. C.; Wang, J.; Guschin, D. Y.; Lee, Y. L.; Rupniewski, I.; Beausejour, C. M.; Waite, A. J.; Wang, N. S.; Kim, K. A.; Gregory, P. D.; Pabo, C. O.; Rebar, E. J. (2007) “An improved zinc-finger nuclease architecture for highly specific genome editing” Nature Biotechnology, 25 (7): 778-785, doi:10.1038/nbt1319, PMID 17603475, the contents of each of which are incorporated herein by reference.

TALENs are artificial restriction enzymes that have a TAL effector DNA-binding domain fused to a DNA cleavage domain. TALENs can also be engineered to target specific DNA sequences. The design and use of TALENs is known in the art and described in, for example, Boch J (February 2011) “TALEs of genome targeting” Nature Biotechnology 29 (2): 135-6, doi:10.1038/nbt.1767. PMID 21301438; Juillerat A, Pessereau C, Dubois G, Guyot V, Maréchal A, Valton J, Daboussi F, Poirot L, Duclert A, Duchateau P (January 2015) “Optimized tuning of TALEN specificity using non-conventional RVDs” Scientific Reports, 5: 8150, doi:10.1038/srep08150. PMC 4311247, PMID 25632877; and Mahfouz M M, Li L, Shamimuzzaman M, Wibowo A, Fang X, Zhu J K (February 2011) “De novo-engineered transcription activator-like effector (TALE) hybrid nuclease with novel DNA binding specificity creates double-strand breaks” Proceedings of the National Academy of Sciences of the United States of America, 108 (6): 2623-8, Bibcode:2011PNAS,108.2623M, doi:10.1073/pnas.1019533108, PMC 3038751, PMID 21262818, the contents of each of which are incorporated herein by reference.

The CRISPR system is a prokaryotic immune system that provides acquired immunity against foreign genetic elements, such as plasmids and phages. CRISPR systems include one or more CRISPR-associated (Cas) proteins that cleave DNA at clustered, regularly-interspersed palindromic repeat (CRISPR) sequences. Cas proteins include helicase and exonuclease activities, and these activities may be on the same polypeptide or on separate polypeptides. Cas proteins are directed to CRISPR sequences by RNA molecules. A CRISPR RNA (crRNA) binds to a complementary sequence in the target DNA to be cleaved. A transactivating crRNA (tracrRNA) binds to both the Cas protein and the crRNA to draw the Cas protein to the target DNA sequence. Not all CRISPR systems require tracrRNA. In nature crRNA and tracrRNA occur on separate RNA molecules, but they also function when contained a single RNA molecule, called a single guide RNA or guide RNA (gRNA). The one or more RNAs and one or more polypeptides assemble inside the cell to form a ribonucleoprotein (RNP). CRISPR systems are described, for example, in van der Oost, et al., CRISPR-based adaptive and heritable immunity in prokaryotes, Trends in Biochemical Sciences, 34(8):401-407 (2014); Garrett, et al., Archaeal CRISPR-based immune systems: exchangeable functional modules, Trends in Microbiol. 19(11):549-556 (2011); Makarova, et al., Evolution and classification of the CRISPR-Cas systems, Nat. Rev. Microbiol. 9:467-477 (2011); and Sorek, et al., CRISPR-Mediated Adaptive Immune Systems in Bacteria and Archaea, Ann. Rev. Biochem. 82:237-266 (2013), the contents of each of which are incorporated herein by reference.

CRISPR-Cas systems have been placed in two classes. Class 1 systems use multiple Cas proteins to degrade nucleic acids, while class 2 systems use a single large Cas protein. Class 1 Cas proteins include Cas10, Cas10d, Cas3, Cas5, Cas8a, Cmr5, Cse1, Cse2, Csf1, Csm2, Csx11, Csy1, Csy2, and Csy3. Class 2 Cas proteins include C2c1, C2c2, C2c3, Cas4, Cas9, Cpf1, and Csn2.

CRISPR-Cas systems are powerful tools because they allow gene editing of specific nucleic acid sequences using a common protein enzyme. By designing a guide RNA complementary to a target sequence, a Cas protein can be directed to cleave that target sequence. In addition, although naturally-occurring Cas proteins have endonuclease activity, Cas proteins have been engineered to perform other functions. For example, endonuclease-deactivated mutants of Cas9 (dCas9) have been created, and such mutants can be directed to bind to target DNA sequences without cleaving them. dCas9 proteins can then be further engineered to bind transcriptional activators or inhibitors. As a result, guide sequences can be used to recruit such CRISPR complexes to specific genes to turn on or off transcription. Thus, these systems are called CRISPR activators (CRISPRa) or CRISPR inhibitors (CRISPRi). CRISPR systems can also be used to introduce sequence-specific epigenetic modifications of DNA, such acetylation or methylation. The use of modified CRISPR systems for purposes other than cleavage of target DNA are described, for example, in Dominguez, et al., Beyond editing: repurposing CRISPR-Cas9 for precision genome regulation and interrogation, Nat. Rev. Cell Biol. 17(1):5-15 (2016), which is incorporated herein by reference.

The agent may be any component of a CRISPR system, such as those described above. For example and without limitation, the CRISPR component may be one or more of a helicase, endonuclease, transcriptional activator, transcriptional inhibitor, DNA modifier, gRNA, crRNA, or tracrRNA. The CRISPR component contain a nucleic acid, such as RNA or DNA, a polypeptide, or a combination, such as a RNP. The CRISPR nucleic acid may encode a functional CRISPR component. For example, the nucleic acid may be a DNA or mRNA. The CRISPR nucleic acid may itself be a functional component, such as a gRNA, crRNA, or tracrRNA.

The agent may include an element that induces expression of the CRISPR component. For example, expression of the CRISPR component may be induced by an antibiotic, such as tetracycline, or other chemical. Inducible CRISPR systems have been described, for example, in Rose, et al., Rapidly inducible Cas9 and DSB-ddPCR to probe editing kinetics, Nat. Methods, 14, pages 891-896 (2017); and Cao, et al., An easy and efficient inducible CRISPR/Cas9 platform with improved specificity for multiple gene targeting, Nucleic Acids Res. 14(19):e149 (2016), the contents of which are incorporated herein by reference. The inducible element may be part of the CRISPR component, or it may be a separate component.

The fluid may contain an excipient. The excipient may facilitate transfer of the agent or analysis or quantification of transfer of the agent. For example and without limitation, the excipient may be 1,2,4,5 benzenetetracarboxylic acid, 3,3′ thiodipropione acid, 8-arm poly(ethylene glycol), adipic acid, alpha-cyclodextrin, cysteine, didodecyl 3,3′-thiodipropionate, EDTA, fructose, glycerin, mannose, mucin, poloxamer 407, poly(lactide glycolide) acid, poly(vinyl alcohol), polyethoxylated castor oil, saccharin, sodium glycolate, sodium glycocholate, sodium taurodeoxycholate, or sodium thiosulfate.

The methods may include introducing fluid and/or an agent to the fluid chamber. The fluid, the agent, or both may introduced into the fluid chamber prior to delivering the ultrasound energy, at the same time as delivering the ultrasound energy, or both before and during delivery of the ultrasound energy.

One or more of the steps described above may be repeated. For example, the methods may include repeating one or more of the introducing and delivering steps. The steps may be performed two, three, four, five, or more times. The steps may be repeated at defined intervals. For example and without limitation, the steps may be repeated at intervals of 5 minutes, 10 minutes, 15 minutes, 30 minutes, 1 hour, 2 hours, 4 hours, 4 hours, 1 day, 2 days, 3 days, 4 days, 5 days, 1 week, 2 weeks, 3 weeks, 4 weeks or more.

The methods may include repeated a method described above according to a schedule. The schedule may include repeated administrations of an agent to buccal tissue at defined intervals for a defined period. For example, schedule may include repeated administration at intervals of about 1 day, about 2 days, about 3 days, about 4 days, about 5 days, or about 7 days over a period of about 1 week, about 2 weeks, about 3 weeks, about 4 weeks, about 6 weeks, about 8 weeks, or about 12 weeks.

The methods may include the use of a device of the invention, such as one of the devices described above in relation to devices of the invention.

The subject may be any type of subject, such as an animal, for example, a mammal, for example, a human. The subject may suffer from a disease, disorder or condition that affects the mouth or for which delivery of an agent to buccal tissue can provide a benefit. For example and without limitation, the disease, disorder, or condition may be or include amalgam tattoo, angular cheilitis, aphthous stomatitis, Bednar's aphthae, Behcet's disease, burning mouth syndrome, cancer, candidiasis, canker sores, cold sores, Crohn's disease, eosinophilic esophagitis, foot-and-mouth disease, gastrointestinal disorders, geographic tongue, gingivitis, gum disease, herpangina, herpes labialis, herpes simplex, infection, inflammatory bowel disease, inflammatory diseases, leukoplakia, lichen planus, mucositis, oral mucositis, pemphigus vulgaris, periodontitis, pharyngitis, side effect of chemotherapy, side effect of radiation therapy, Sjogren's syndrome, Sutton's disease, temporomandibular joint syndrome, transient lingual papillitis, ulcers, ulcerative colitis, or xerostomia.

### EXAMPLES

### Example 1: Ultrasound Delivery to Treat Ulcer in Hamsters

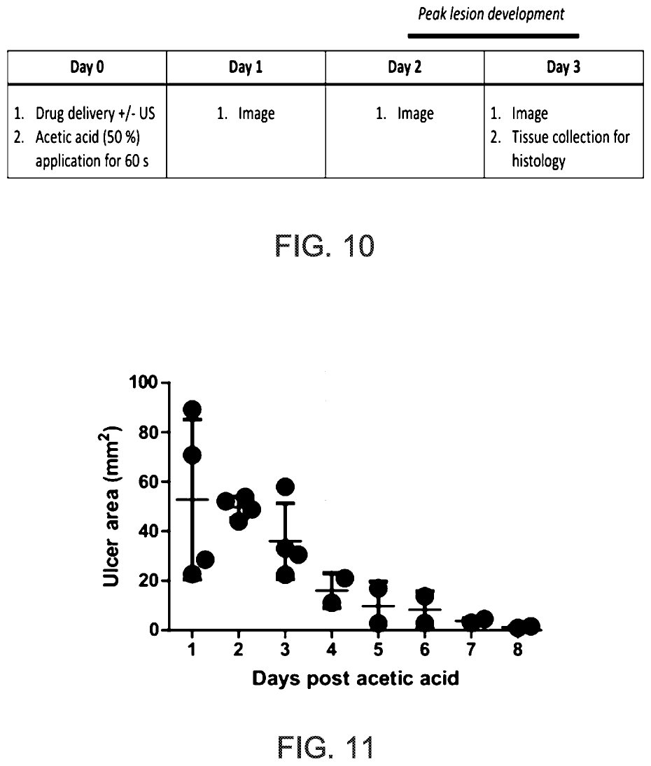

FIG. 10 is a schematic of the procedure used to test delivery of budesonide to treat or prevent ulcers induced by acetic acid in hamster cheek pouches. 50% acetic acid was administered for 60 seconds.

FIG. 11 is a graph of ulcer area at various time points after administration of acetic acid to hamster cheek pouches.

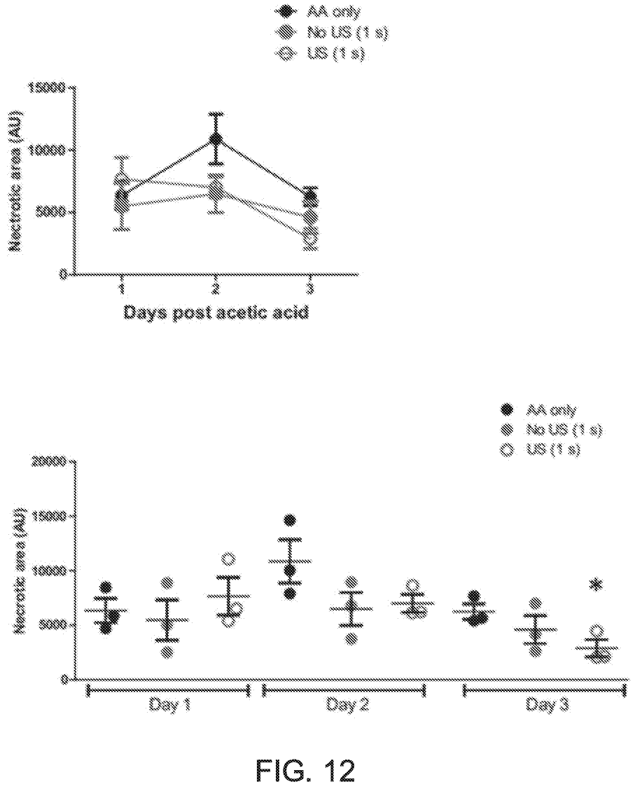

FIG. 12 shows graphs of necrotic area at various time points after administration of acetic acid to hamster cheek pouches. Animals were treated with no budesonide, budesonide in the absence of ultrasound, or budesonide in the presence of ultrasound, as indicated.

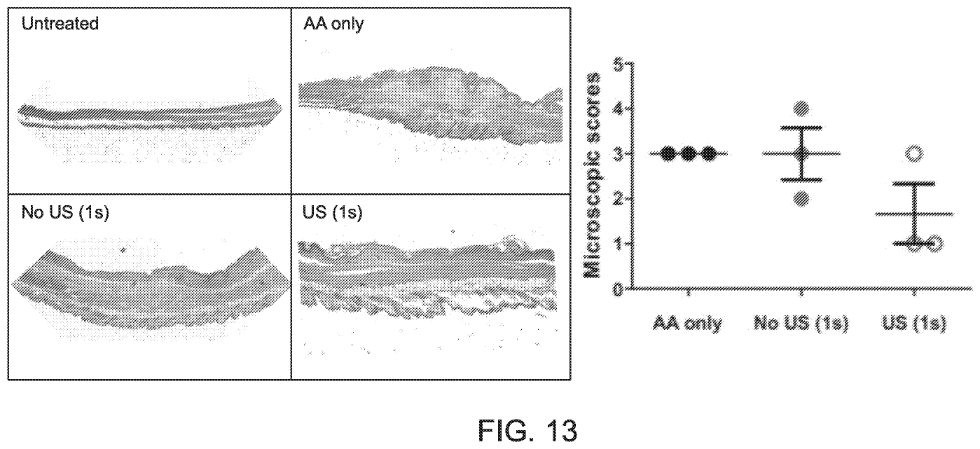

FIG. 13 shows histopathological images of hamster cheek pouches after administration of acetic acid and graph of microscopic scores of the histopathological images. Animals were treated with no budesonide, budesonide in the absence of ultrasound, or budesonide in the presence of ultrasound, as indicated.

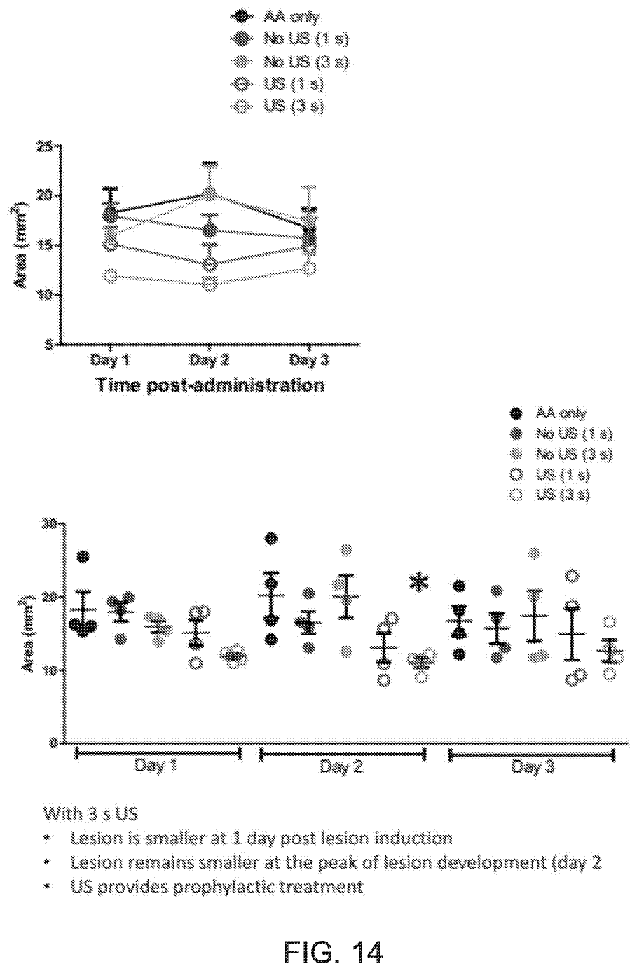

FIG. 14 shows graphs of ulcer area at various time points after administration of acetic acid to hamster cheek pouches. Animals were treated with no budesonide, budesonide in the absence of ultrasound for 1 second, budesonide in the absence of ultrasound for 3 seconds, budesonide in the presence of ultrasound for 1 second, or budesonide in the presence of ultrasound for 3 seconds, as indicated.

### Example 2: Abstract

The delivery of therapeutics to the gastrointestinal (GI) mucosa remains primarily a function of diffusion and rapid delivery is a significant goal in drug delivery science. However, delivery is hindered by the molecular barrier properties of the mucosa, as well as environmental factors. Delivery to the oral cavity, in particular, is limited by mastication and salivation, limiting drug absorption further. We hypothesized that low-frequency ultrasound can overcome these barriers and achieve rapid, localized delivery independent of diffusion. We developed a device compatible with buccal therapeutic administration. The device creates a drug reservoir against the mucosa through which 40 kHz ultrasound is applied. Performance was characterized and found to generate sufficient pressures as to generate transient cavitation while minimizing thermal effects. Delivery of fluorescently-labeled dextrans with molecular weights between 3 and 500 kDa was quantified using fresh porcine small intestine ex vivo. Short, 60-second treatments facilitated rapid penetration of the dextrans independent of molecular weight. Finally, we investigated the capacity of the form-factor to treat oral inflammatory lesions in hamster cheek pouches using budesonide given the drug's broad clinical use. Ultrasound-mediated administration was found to have a prophylactic effect on oral lesion size, reducing initial lesion size compared to topical budesonide alone. We demonstrate the utility of this technology for ultra-rapid delivery to the oral cavity. The technology dramatically enhances the efficacy of known anti-inflammatory substances, such as budesonide, by maximizing penetration, while providing a means for local administration of larger molecules. The capacity to deliver a broad range of therapeutics, including macromolecules, presents an intriguing capability that may further expand the repertoire of therapeutics that can be applied topically in the mouth and beyond.

### Example 3: Introduction

The ability to facilitate the rapid and targeted delivery of a therapeutic directly into tissue independent of diffusion and drug-tissue contact time has been a major aim of the drug delivery field. Indeed, enabling ultrarapid, and targeted drug administration to the gastrointestinal (GI) tract has been a long-standing goal given the great potential for treating a myriad of diseases such as inflammatory bowel disease (IBD) [1, 2]. In patients and in rodent models of IBD, local high drug concentrations at the site of disease within the GI tissue have been demonstrated to afford better therapeutic outcomes [3, 4]. However, therapeutic delivery to the GI tract remains an area of intense research owing to the challenges presented by the physiology of the GI tract itself [5]. For example, the GI mucosa functions as a striking barrier to drug absorption, greatly limiting passive diffusion [6]. In addition, the wealth of proteases and nucleases present locally further complicates the mucosal delivery of therapeutics, particularly biologics or nucleic acids [4, 6]. Current strategies for targeted delivery of therapeutics focus on formulation-based approaches [5, 7-9] but are limited by the requirement for tedious drug-specific formulation, intravenous administration, potentially fatal side effects [10], and minimal long-term tissue retention [11]. We have recently reported on the use of ultrasound as a drug delivery method in the GI tract [4, 12]. Ultrasound is a sound wave with a frequency greater than the audible range of humans (>20 kHz) [13]. In the clinical setting, a broad range of applications using ultrasound have been utilized including imaging, lithotripsy, liposuction, and tumor ablation [14]. Ultrasound has been proposed as an effective and safe transdermal drug delivery method [15] and has gained FDA approval for transdermal delivery of lidocaine [16]. Specifically, low frequency ultrasound (≤100 kHz) can be used as a physical enhancer to facilitate therapeutic delivery by leveraging the phenomenon of transient cavitation, in which micron-scale bubbles are nucleated and subsequently collapse, creating a jet of fluid, to physically propel therapeutics into the tissue [16, 17]. We have demonstrated safety and tolerability of using ultrasound in the GI tract of both pigs and mice [12]. In addition, we have demonstrated the capability to deliver naked nucleic acids for the reduction in disease severity in a mouse model of experimental colitis [4]. In these studies, the therapeutic and ultrasound was simultaneously administered locally in the colon [4]. In the setting of IBD, the colon is filled with a medicated enema and ultrasound applied through the fluid. This mode would not be feasible in other areas of the GI tract, such as the oral cavity, and would require a technology to have a self-contained drug reservoir that could be applied locally on the mucosa. To the best of our knowledge, such an embodiment has not been engineered previously.

A technology that facilitates rapid, local administration in the oral cavity independent of drug-tissue contact time could have a profound positive impact on treatment paradigms for diseases of the oral cavity [18]. Mastication and salivation greatly diminish drug-tissue contact time, and therefore, absorption, complicating outcomes [19]. In the setting of head and neck cancer, for example, treatment-limiting oral inflammation is common and leads to differential treatment spending between $17,000-$25,000 per patient [20, 21]. Patients whose oral mucositis cannot be managed by this extensive intervention typically discontinue radiation for a week, which increases the risk of cancer recurrence, shortens recurrence-free survival, and results in inferior overall survival [22]. The oral component of Crohn's disease is another debilitating example where patients today resort to compounded mouth rinse solutions using corticosteroids [23]. Poorly controlled oral inflammation often requires emergency medical intervention and supplemented nutrition in a clinical setting because eating can become too painful for those patients [24]. The efficacy of currently-utilized therapies, such as corticosteroids, might be dramatically enhanced by maximizing local uptake at the site of disease [18].

Based on these considerations, we aimed to engineer a system that could be applied in the mouth and facilitate rapid, local delivery of therapeutics and assessed its efficacy in an oral injury model. In the present studies, we applied low-frequency ultrasound in a form-factor amenable to administration in the oral cavity for the treatment of oral inflammatory lesions in hamster cheek pouches [25]. We chose to focus on the administration of budesonide, a corticosteroid, given its clinical relevance in treating a range of inflammatory diseases.

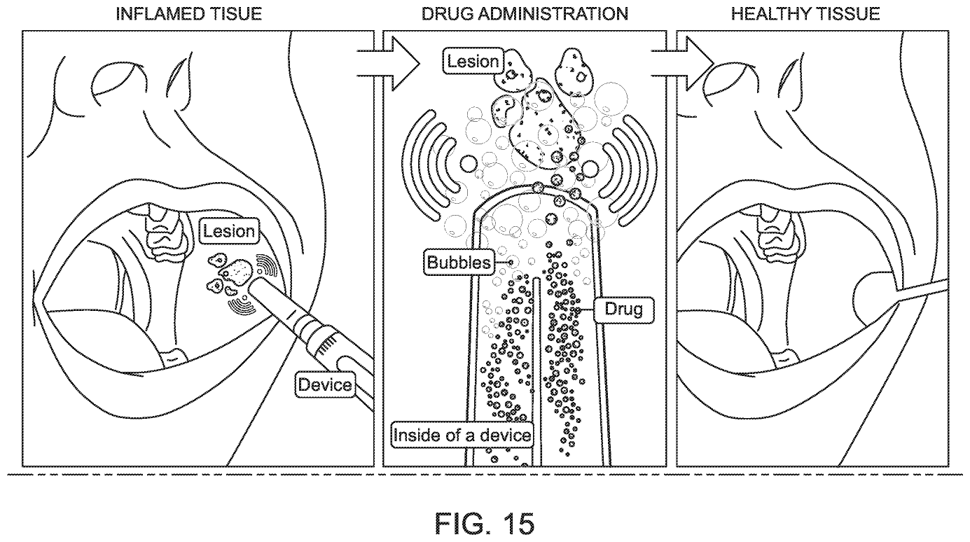

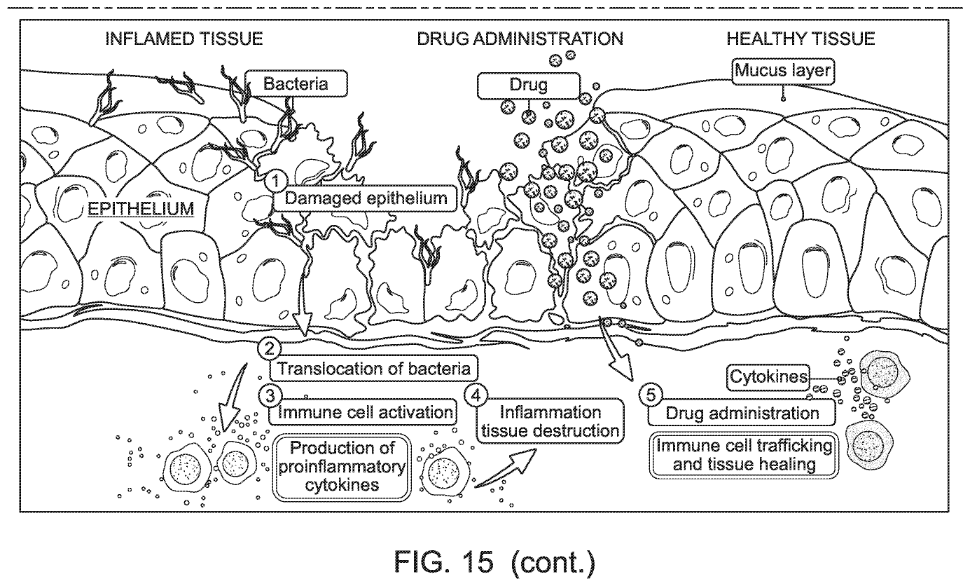

FIG. 15 is a graphic abstract of the ultrasound devices and methods for treatment of oral lesions according to an embodiment of the invention.

### Example 4: Materials and Methods

Chemicals and Drug Preparation Dextran, Alexa Fluor™ 680, 3 kDa (D34681) was purchased from Thermo Fisher Scientific (US). Cy5.5 labeled dextrans, 10 kDa (DX10-S55-1) and 500 kDa (DX500-S55-1) were purchased from Nanocs Inc. (US). Budesonide (B7777), 2-hydroxypropyl-β-cyclodextrin (HP-β-CD, H107), and glacial acetic acid (ARK2183) were purchased from Sigma Aldrich. Budesonide (0.1% w/v) was prepared in deionized water containing HP-β-CD (7% w/v). Suspensions were vortexed quickly and placed in an ultrasonic bath (42° C.) for 1.5-2.5 hour until solubilized. Budesonide:HP-β-CD solutions were prepared fresh for each experiment and were stored at −20° C. until use. Acetic acid (50%) solution was prepared fresh in deionized water for each experiment.

Ultrasound Device

The device housing was designed to allow for controlled application on oral mucosa. A 40 kHz CV401 converter (Sonics & Materials, Newtown, Conn.) was utilized for ultrasound generation. The converter was coupled to a custom-built aluminum half-wave horn with a linear taper to focus the acoustic energy while keeping the overall length amenable to hand-held use. The housing was designed to fix the tissue-aluminum horn tip distance to 3 mm to provide reproducible ultrasound exposure. This housing was 3D-printed using clear resin from Formlabs, Inc. (Somerville, Mass.) (FIG. 1a). Aqueous solutions were introduced into the fluid chamber using a fluid channel fitted with a Luer fitting for compatibility with a syringe.

The 3D acoustic pressure field produced by the device was mapped in a water tank using a needle hydrophone (Precision Acoustics, UK) whose position was set by motor-controlled stages (Velmex Inc., Bloomfield, N.Y.). A three-dimensional space was mapped in 0.5 mm increments utilizing control software to move the hydrophone, trigger the ultrasound probe, and record a measurement utilizing VirtualBench and a custom LabVIEW program (National Instruments, Austin, Tex.). Three amplitude measurements were recorded at each position and averaged. This signal was then converted to kilopascal and 3D pressure maps plotted using MATLAB (MathWorks, Natick, Mass.).

Ex Vivo Delivery and Quantification of Fluorescently-Labeled Dextrans to Porcine Mucosa

Fresh porcine small intestinal tissue was harvested from Yorkshire pigs after sacrifice and used within four hours of procurement. Tissues were rinsed, trimmed, and opened into circular segments using a 1.25-inch biopsy punch; the ultrasound device was positioned firmly on top of the mucosal surface to prevent leakage. Subsequently, the fluid chamber was filled with a solution of fluorescently-labeled dextran at a concentration of 0.5 (3k kDa) or 1 μg/mL (10 and 500 kDa) prepared in phosphate-buffered saline (PBS). Ultrasound was applied for 60 seconds at an intensity of 5 W/cm2 (calibrated by calorimetry) and a 50% duty cycle to reduce thermal effects [26]. During ultrasound treatment, the temperature was measured at the serosal tissue surface and recorded every second using a digital thermometer with a sampling rate of 2.5 times per second (Traceable® type K thermometer, VWR, Radnor, Pa.). All experiments were performed at room temperature.

Immediately following ultrasound delivery, tissues were thoroughly washed to remove residual dextran and imaged to quantify fluorescent intensity. Tissues were imaged using an Azure c600 Imaging System (Azure Biosystems, Inc., Dublin, Calif.). The IR channel (660 nm) was utilized with an exposure time of 500 ms at a resolution of 60 μm. The fluorescence intensity for each sample was calculated by subtracting the fluorescence measured from the periphery of the tissue (autofluorescence) from the total fluorescence measured within the region where the device and dextran were applied

Application to Dogs In Vivo

All animal-related research aspects were approved and conducted in accordance with the ethical standards and guidelines of the Institutional Animal Care & Use Committee (IACUC) at Tufts University. The tolerability of the oral ultrasound device was tested in adult beagles. Beagles were chosen because they can be trained to accept various procedures, making anesthesia unnecessary. Because the animals are conscious, their behavior and auditory cues may be monitored to assess potential tolerability of the device. Five adult beagles were selected at random by veterinary staff for this study. Dogs were brought into the procedure room individually and allowed to move around freely. The device was initially run in a beaker of fluid at the same power described above to monitor the animal's reaction to the ultrasonic noise. While running in the beaker of fluid, the device was slowly brought closer to the animal and their behavior monitored by veterinary staff.

To apply the device to the oral cavity, animals were placed on a risen platform and lightly restrained by veterinary staff to encourage them to lie on their left sides, allowing for application of the device to the right cheek. Before treatment began, an image was captured of the mucosal region to be treated. The device was placed against the cheek and the fluid chamber filled with water. The device was turned on for 5 seconds at a duty cycle of 100% and the animal's reaction and behavior were monitored by the veterinary staff. After treatment, the device was removed and the treated area gently blotted dry. A second image was taken of the treated area. The animals were then offered treats and taken out of the room.

In Vivo Treatment of Oral Inflammatory Lesions in Hamster Cheek Pouches

Male Golden Syrian hamsters weighing 100-120 g (Charles River Laboratories, Wilmington, Mass.) were used as received and randomly assigned to experimental groups by the researchers performing the work. To limit pain and distress, all animals received buprenorphine sustained release (SR) (subcutaneous administration, 1 mg/kg body weight, compounded by ZooPharm) just prior to the application of acetic acid for the induction of the oral lesion on day 0.

All manipulations occurred with the animal under general anesthesia by inhaled isoflurane. Experimental treatment groups included topical application of budesonide solution with (Budesonide+US) and without (Budesonide) ultrasound. A positive disease control receiving no treatment was also included (Disease). Treatment was administered on day 0 immediately prior to lesion induction using acetic acid. While under general anesthesia, the left cheek pouch was everted, the device was placed on the buccal surface, and the fluid chamber filled with solubilized budesonide solution (0.1%, ˜1 mL). For the Budesonide+US treatment group, ultrasound was simultaneously applied for 3 seconds at an intensity of 5 W/cm2 and a 100% duty cycle. In experimental groups receiving budesonide only (Budesonide), drug was applied as described above without turning the ultrasound on. Immediately following budesonide treatment, oral lesions were induced by topical application of acetic acid (50% solution) as described in previous studies [27-29]. Briefly, acetic acid was applied to the buccal surface using a liquid holder with a circular area of 5 mm2 for 60 seconds. After acetic acid application, the hamsters were placed back into their cages to recover. On every subsequent day, the cheek pouches were everted to reveal the lesion and an image captured to quantify lesion size. Treatments were then applied as described above. Lesion size was measured using ImageJ (National Institutes of Health, Bethesda, Md.).

Statistics

All data analysis was performed using GraphPad Prism software (GraphPad Software, Inc., La Jolla, Calif., USA). Two-group comparisons were made using Student's unpaired t-test using Welch's correction. Multiple comparisons were made using one-way ANOVA followed by Tukey's post-test. For all comparisons, p<0.05 was considered statistically significant. Data are reported as mean±standard error of the mean (SEM) except for temperature measurements that are reported as mean±standard deviation (SD).

### Example 5: Results

Characterization of the Oral Device

The handheld oral ultrasound device was designed to easily and rapidly deliver an aqueous therapeutic agent to a mucosal epithelial surface. The device developed as part of this study houses the ultrasound converter and horn and contains a fluid chamber with a maximum fill volume of 2 mL of a medicated fluid.

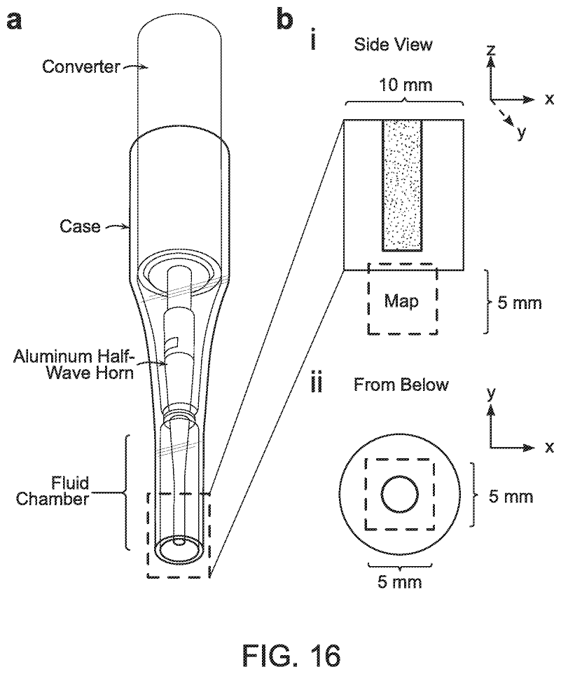

FIG. 16 is an illustration of the oral ultrasound device for local administration of therapeutics to mucosal surfaces. Part a of the figure shows that the oral ultrasound device prototype consists of an aluminum half-wave horn (5 mm diameter) fitted in a case that includes a fluid chamber that is filled with a therapeutic solution (˜1 mL). For application, the device is applied with the opening of the fluid chamber positioned against the mucosa and 40 kHz ultrasound is emitted through the solution, facilitating the delivery of the therapeutic agent into the tissue. Part b of the figure shows pressure mapping region for the horn within the case as viewed from i) the side, and ii) the bottom.

A linear-tapered horn with a working distance of 3 mm delivers 40 kHz frequency ultrasound to an epithelial surface at a power of 5 W/cm2, a relevant intensity within FDA guidelines for energy-emitting devices. A 3D pressure map acquired in a water tank demonstrates the range and depth of ultrasound-mediated pressure for the device.

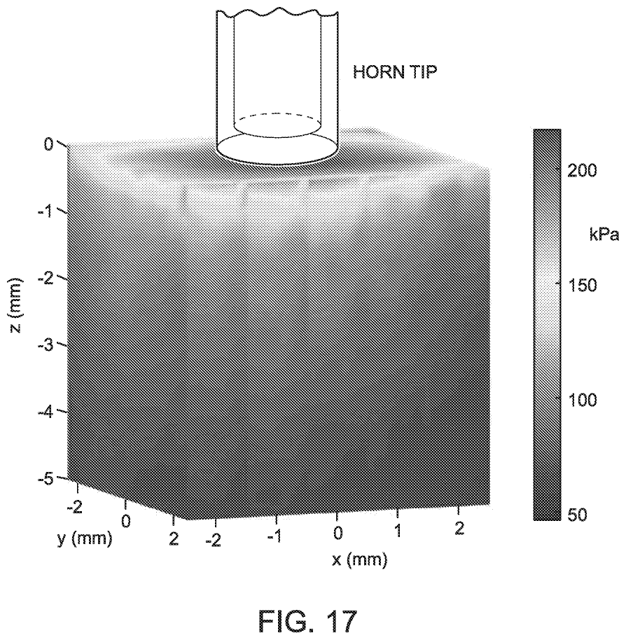

FIG. 17 shows a 3D acoustic pressure map of the horn in FIG. 16 with the fluid chamber fully submerged in water operating at 60% driving amplitude. The color scale ranges from 50 to 260 kPa.

A pressure reading of approximately 150, 100 or 80 kPa is evident at 0.5, 2 and 3.5 mm, respectively, below the outer case surface, the relevant field through which cavitation is generated when the device is applied to tissue [30].

The Oral Device Significantly Enhances the Delivery of Macromolecules Ex Vivo

To determine the delivery capacity of the device, fluorescently-labeled dextrans with molecular weights spanning two orders of magnitude were utilized.

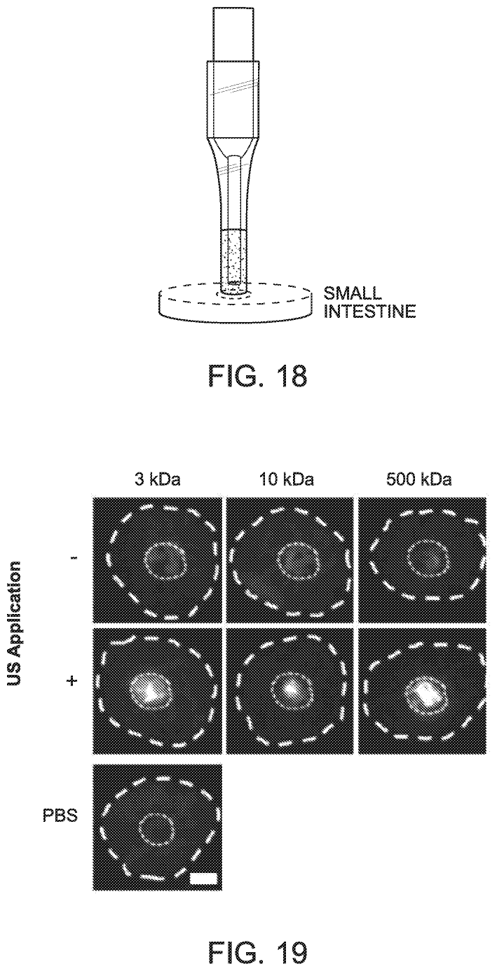

FIG. 18 is a schematic demonstrating delivery of fluorescently-labeled dextrans to small intestinal porcine biopsy punches using the oral ultrasound device. The outer dashed black line outlines the tissue punch while the smaller dashed line indicates where the device was applied. The fluid chamber is loaded with solution containing fluorescently-labeled dextrans (˜1 mL).

Short, 60-second treatments significantly enhanced delivery of all dextran sizes when compared to delivery without ultrasound (3 kDa, p=0.0003; 10 kDa, p=0.02; and 500 kDa, p=0.0007) as measured by fluorescence intensity and increased 4 to 5.5-fold with ultrasound.

FIG. 19 shows representative fluorescent images of porcine mucosal tissue after delivery of dextrans of various molecular weights (3, 10, and 500 kDa) with (+) and without (−) ultrasound application for 60 seconds. PBS with ultrasound served as a control. The entire tissue is encircled (thicker dashed white line) and delivery of labeled dextrans are observed in red at the site of application (smaller dashed white line). Scale bar is 1 cm.

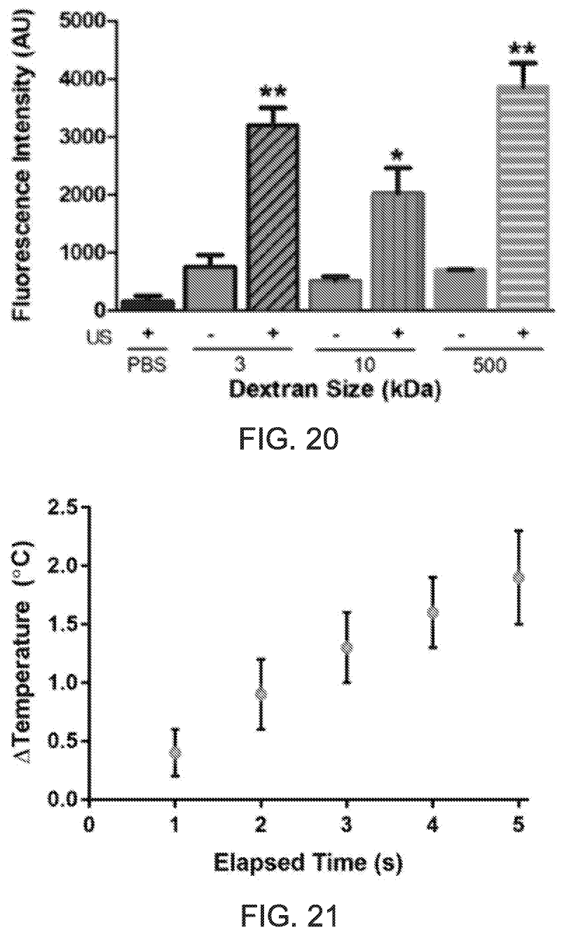

FIG. 20 is a graph of fluorescence intensity of the porcine mucosa measured immediately after delivery (n=3-6). Data are presented as mean±SEM. p values were determined by two-tailed, unpaired t-test using Welch's correction; * p<0.05, ** p<0.001.

During ultrasound treatment, the tissue temperature increased 1.3° C. in the first 3 seconds of application and approximately 12° C. over the entire 60 second treatment (21.9±1.04° C. to 34.0±1.41° C., n=36).

FIG. 21 is a graph of the temperature rise during ultrasound treatment on porcine small intestine with a 50% duty cycle (n=36). The graph shows the temperature rise during the first 5 seconds, a representative time range for use in treating oral mucosa. Data are presented as mean±1 SD.

Application in Dogs is Well Tolerated

We next aimed to determine the tolerability and sensation associated with use of the oral ultrasound device. This was tested in awake, unanesthetized dogs. Dogs were chosen because interspecies comparisons of oral mucosal bioavailability indicate great similarity between dogs and humans [31] and because dogs can be trained to accept administration without the use of anesthesia, allowing for observation of their reaction to the device. All animals appeared unperturbed by the noise generated by the device, regardless of the distance of the device to the animal in the room. Because the noise appeared to pose no issue, application in the mouth was tested. During treatment, animals did not show enhanced levels of stress as determined by the veterinary staff. Additionally, there were no changes in appearance to the oral mucosa after treatment and all animals accepted treats ad libitum upon completion of treatment.

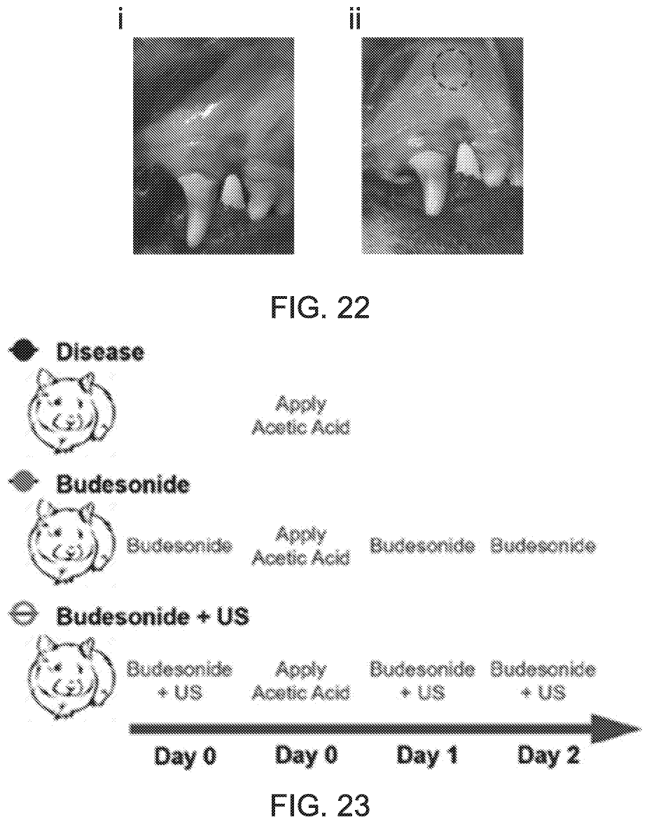

FIG. 22 shows representative images of the oral mucosa of dogs i) before and ii) after ultrasound treatment. The dotted line indicates the area of the mucosa that was treated.

Ultrasound-Mediated Budesonide Delivery Lessens Oral Lesion Severity In Vivo

Following characterization of the oral ultrasound device and its tolerability in large animals, we investigated its efficacy in an animal model of oral inflammatory lesions.

FIG. 23 is a schematic of the three experimental groups and respective disease induction and treatment regimen using ultrasound-mediated budesonide delivery to treat oral lesions in hamster cheek pouches in vivo.

The most frequently used model for oral lesions is the hamster buccal pouch model [28] and is amenable to the size of the device. Previous histological evaluation of oral lesions induced by acetic acid has validated the presentation of clinically relevant signs, including ulceration, necrosis with suppurative inflammation, edema, and infiltration of neutrophils [25, 28]. In our studies, oral lesions induced by topical application of acetic acid solution (50%) for 60 seconds resulted in round, elevated white lesions approximately 24 hours after acetic acid application.

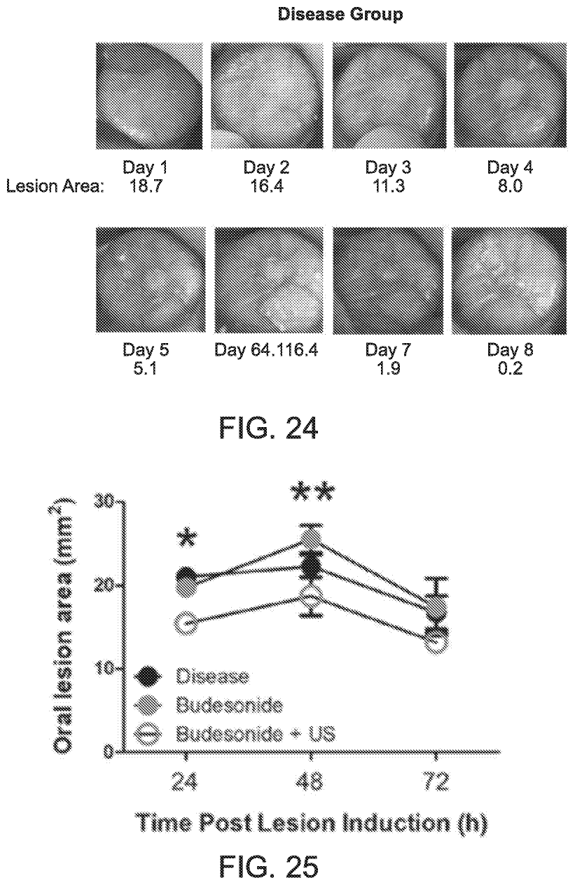

FIG. 24 shows representative images of oral lesions in the disease group (untreated) over an 8-day period. Lesions were imaged daily after lesion induction and the lesion area is indicated in square millimeters.

Early injury was characterized by vasoconstriction within- and vasodilation in vessels surrounding the area of application after day 1. Days 2 and 3 were characterized by mounting edema, and congestion of the blood vessels surrounding the lesion. Further, these changes were accompanied by at least partial central necrosis surrounded by granulation tissue at the interface zone towards the surrounding normal epithelium. By day 4, all lesions appeared pus-filled, constricted and remained well-circumscribed. It is noteworthy that the lesion remained well circumscribed and corresponded to the initial site of injury. Lesions nearly self-resolve by day 8 post injury.

In order to evaluate whether ultrasound-mediated delivery of budesonide compared to topical application is more effective in the treatment of oral lesions, the first 72 hours following injury induction were analyzed because peak severity occurs during this period. In disease animals that did not receive any budesonide treatment, oral lesion reached a maximum size 48 hours following topical application of acetic acid and begin to heal naturally after 72 hours.

FIG. 25 is a graph showing that oral lesion areas remain smaller in the budesonide+US treatment group following 24 and 48 h following lesion induction with acetic acid (n=17 for 24 and 48 h; n=4 for 72 h).

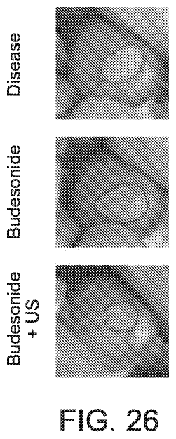

FIG. 26 shows representative images of macroscopic oral lesion areas at 48 h post acetic acid application, * p<0.05 vs. disease and budesonide ** p<0.05 vs. budesonide, One-way ANOVA Tukey post-test.

When compared to Disease and Budesonide groups, animals that received Budesonide+US exhibited smaller oral lesion sizes 24 hours post-acetic acid induced injury (Disease, 20.5+/−1.1 mm2; Budesonide, 20.3+/−1.1 mm2; Budesonide+US, 15.7+/−1.3 mm2; p<0.05). These results demonstrate that budesonide in combination with ultrasound reduces the overall severity and initial size of the lesion using a single dose in a prophylactic manner. Following 48 hours after acetic acid-induced injury, oral lesions in hamster cheek pouches treated with budesonide in combination with ultrasound remained smaller than those treated topically with budesonide without ultrasound (Budesonide+US, 18.71+/−2.3 mm2; Budesonide, 25.5+/−1.6 mm2, p<0.05). After 72 hours, oral lesions began to heal naturally and oral lesions in all groups were similar in size. These results demonstrate that budesonide in combination with ultrasound confines the size of the oral lesion, thus preventing the lesion from attaining the severity observed in the Disease and Budesonide groups.

### Example 6: Discussion

Presently, we've developed and tested an ultrasound device that can be used for targeted delivery in the oral cavity for the treatment of oral lesions. Ultrasound has been used in a broad range of applications in the clinic including imaging, lithotripsy, liposuction, and tumor ablation [1, 17]. The frequency range, intensity, and beam shape for the medical use of ultrasound varies upon application. Ultrasound employed for diagnostic imaging typically uses low intensity, high frequency wavelengths, in the 2-10 MHz range [32]. In contrast, skin sonophoresis (also called phonophoresis) utilizes ultrasound with a shorter wavelength, 20-100 kHz, resulting in the slower dissipation and deeper penetration of the acoustic pressure wave [13]. Transient cavitation is the predominant mechanism by which low frequency ultrasound is able to permeabilize tissue [33]. Compared to the skin, the barrier function of GI mucosa is lower, allowing for shorter treatment times, and, as a result, simultaneous application of the therapeutic and the ultrasound [12]. In this scenario, transient cavitation events act to physically propel the therapeutic into the tissue, enabling high local concentrations of drug within the tissue and the ability to deliver larger molecular weight therapies [4]. Indeed, these capabilities might allow for near term clinical translation of the technology for use with traditional small-molecule therapies whose efficacy is currently limited by delivery and passive diffusion, such as in the case of corticosteroids for treating oral inflammation as we note here, or the use of rectal mesalamine in the setting of ulcerative colitis [3, 34]. Long term, this formulation-independent delivery may enable the use of novel therapeutics, such as topical proteins or nucleic acids, for highly-targeted treatments directly at the site of disease. Clinical translation of this technology is now dependent on the creation of relevant form-factors capable of meeting FDA requirements.

Here we developed a hand-held device that applies low frequency ultrasound at 40 kHz through a liquid filled chamber to enhance drug delivery to apposing oral tissue (FIG. 1a). In this study, the ultrasound device was successfully tested on several mucosal surfaces including porcine intestine ex vivo, and canine and hamster oral mucosa in vivo, but other uses, such as transdermal treatment, are possible. The device significantly enhances the delivery of large molecular weight molecules, which are typically incapable of passive diffusion because of their size [35]. The results presented here further corroborate our previous studies in the colon demonstrating the capacity of ultrasound to facilitate rapid delivery of a wide-range of therapeutics, including nucleic acids, proteins and small molecule drugs like mesalamine [4, 12]. The short treatment times required have the added benefit of limiting potential thermal effects, which can be observed when utilizing extended exposure times.

Another important step on the path to translation is the tolerability and sensation induced using such a technology. Herein represents the first report of ultrasound application in an awake, unsedated large-animal model. We found dogs tolerated placement and application of the device in the mouth. Based on behavioral observation of the animal, the sensation and sound emitted by the device were deemed to pose no issue for the animals. This is a crucial step on the path to translation of a combination product, demonstrating safe use in large animal systems.

Finally, we aimed to demonstrate a more effective treatment for oral lesions in vivo over topical application of a commonly prescribed medication using this technology. Oral lesions are a common and painful condition that affects the mucosal oral cavity. The underlying pathobiology of oral ulcers involves infiltration of inflammatory cells to the ulceration, generation of reactive oxidative species, and the release of pro- and anti-inflammatory cytokines at the site of mucosal damage [36-38]. Thus, current treatments include anti-inflammatory drugs and antiseptics administered as mouthwashes or topical gels [39]. A challenge to these therapeutic formulations is contact time and so treatments often must be administered multiple times a day. With our oral ultrasound device, ultrasound is used to rapidly deliver therapeutics into the inflamed tissue in a short period of time, promoting therapeutic benefit.

In our studies, we employed the hamster buccal pouch model—a common animal model of oral mucosal lesions. The model is commonly used to study the pathobiology of oral lesions and for testing the efficacy of potential therapeutics by which lesions are experimentally induced by acetic acid application, chemotherapy agents, as well as radiation [27-29, 40, 41]. In our studies, a small volume of 50% acetic acid was applied to the oral mucosal surface resulting in a single discrete lesion that was similar in appearance to those described clinically as round, with a necrotic center surrounded by an erythematous (redness of the skin) ring. The application of acid generated a unified size of ulcer [41], which was essential to our studies in which we assessed the anti-inflammatory healing effect of budesonide. Moreover, the pathobiology associated with acetic acid induced lesions is similar to stomatitis in that they both present characteristic inflammatory cell infiltration around the lesion [42]. We hypothesized that through ultrasound-mediated delivery of therapeutics locally via our oral ultrasound device, we would observe a therapeutic benefit compared to the current topical application as a result of rapid delivery into the tissue. Indeed, peak severity was significantly reduced in those animals receiving budesonide in combination with ultrasound, with topical budesonide alone having no beneficial effect. This underscores the importance of local tissue drug concentrations, which is a crucial factor for the overall efficacy of treatment. Indeed, if therapeutic levels are not achieved locally in a given administration using conventional mouthwashes, for example, then the adverse events associated with low-dose systemic corticosteroid administration must be considered. By utilizing the oral ultrasound device, however, a therapeutically-relevant dose of budesonide is rapidly loaded into the tissue, leading to less severe disease.

In summary, we have demonstrated a novel ultrasound device that can enhance mucosal drug delivery. The device is tolerable in an awake animal model and the application of anti-inflammatory budesonide in an acetic-acid hamster buccal pouch injury model confirmed a prophylactic effect resulting from enhanced drug delivery. Enhancing the regional application of therapeutics by maximizing penetration may include clinical applications going beyond regional mucosal injury.

### Example 7: References

1. Traverso, G., et al., Convergence for Translation: Drug-Delivery Research in Multidisciplinary Teams. Angew Chem Int Ed Engl, 2018. 57(16): p. 4156-4163.
2. Maloney, E. and J. H. Hwang, Intra-luminal focused ultrasound for augmentation of gastrointestinal drug delivery. Annals of Translational Medicine, 2017. 5(7).
3. Frieri, G., et al., Mucosal 5-aminosalicylic acid concentration inversely correlates with severity of colonic inflammation in patients with ulcerative colitis. 2000. 47(3): p. 410-414.
4. Schoellhammer, C. M., et al., Ultrasound-Mediated Delivery of RNA to Colonic Mucosa of Live Mice. Gastroenterology, 2017. 152(5): p. 1151-1160.
5. Kamaly, N., et al., Degradable Controlled-Release Polymers and Polymeric Nanoparticles: Mechanisms of Controlling Drug Release. Chem Rev, 2016. 116(4): p. 2602-63.
6. Mitragotri, S., P. A. Burke, and R. Langer, Overcoming the challenges in administering biopharmaceuticals: formulation and delivery strategies. Nat Rev Drug Discov, 2014. 13(9): p. 655-72.
7. McLeod, A. D., D. R. Friend, and T. N. Tozer, Glucocorticoid-dextran conjugates as potential prodrugs for colon-specific delivery: hydrolysis in rat gastrointestinal tract contents. J Pharm Sci, 1994. 83(9): p. 1284-8.
8. McKeage, K. and K. L. Goa, Budesonide (Entocort EC Capsules): a review of its therapeutic use in the management of active Crohn's disease in adults. Drugs, 2002. 62(15): p. 2263-82.
9. Bakhru, S. H., et al., Oral delivery of proteins by biodegradable nanoparticles. Adv Drug Deliv Rev, 2013. 65(6): p. 811-21.
10. Van Assche, G., et al., Progressive multifocal leukoencephalopathy after natalizumab therapy for Crohn's disease. N Engl J Med, 2005. 353(4): p. 362-8.
11. Lamprecht, A., U. Schafer, and C. M. Lehr, Size-dependent bioadhesion of micro- and nanoparticulate carriers to the inflamed colonic mucosa. Pharm Res, 2001. 18(6): p. 788-93.
12. Schoellhammer, C. M., et al., Ultrasound-mediated gastrointestinal drug delivery. Sci Transl Med, 2015. 7(310): p. 310ra168.
13. Polat, B. E., et al., Ultrasound-mediated transdermal drug delivery: mechanisms, scope, and emerging trends. J Control Release, 2011. 152(3): p. 330-48.
14. Miller, D. L., et al., Overview of therapeutic ultrasound applications and safety considerations. J Ultrasound Med, 2012. 31(4): p. 623-34.
15. Prausnitz, M. R. and R. Langer, Transdermal drug delivery. Nat Biotechnol, 2008. 26(11): p. 1261-8.
16. Polat, B. E., D. Blankschtein, and R. Langer, Low-frequency sonophoresis: application to the transdermal delivery of macromolecules and hydrophilic drugs. Expert Opin Drug Deliv, 2010. 7(12): p. 1415-32.
17. Schoellhammer, C. M. and G. Traverso, Low-frequency ultrasound for drug delivery in the gastrointestinal tract. Expert Opin Drug Deliv, 2016. 13(8): p. 1045-8.
18. Sonis, S. T., Ultrasound-mediated drug delivery. Oral Dis, 2017. 23(2): p. 135-138.
19. Rathbone, M. J., S. Senel, and I. Pather, Oral Mucosal Drug Delivery and Therapy Preface. Oral Mucosal Drug Delivery and Therapy, 2015: p. Vii-X.
20. Nonzee, N.J., et al., Evaluating the supportive care costs of severe radiochemotherapy-induced mucositis and pharyngitis: results from a Northwestern University Costs of Cancer Program pilot study with head and neck and nonsmall cell lung cancer patients who received care at a county hospital, a Veterans Administration hospital, or a comprehensive cancer care center. Cancer, 2008. 113(6): p. 1446-52.
21. Elting, L. S., et al., Risk, Outcomes, and Costs of Radiation-Induced Oral Mucositis Among Patients With Head-and-Neck Malignancies. International Journal of Radiation Oncology*Biology*Physics, 2007. 68(4): p. 1110-1120.
22. Ohri, N., et al., Radiation Therapy Noncompliance and Clinical Outcomes in an Urban Academic Cancer Center. International Journal of Radiation Oncology⋅Biology⋅Physics, 2016. 95(2): p. 563-570.
23. Lankarani, K. B., G. R. Sivandzadeh, and S. Hassanpour, Oral manifestation in inflammatory bowel disease: A review. World Journal of Gastroenterology, 2013. 19(46): p. 8571-8579.
24. Asquith, P., R. A. Thompson, and W. T. Cooke, Oral manifestations of Crohn's disease. Gut, 1975. 16(4): p. 249-254.
25. Logan, R. M., et al., The role of pro-inflammatory cytokines in cancer treatment-induced alimentary tract mucositis: pathobiology, animal models and cytotoxic drugs. Cancer Treat Rev, 2007. 33(5): p. 448-60.
26. Schoellhammer, C. M., et al., Rapid skin permeabilization by the simultaneous application of dual frequency, high-intensity ultrasound. J Control Release, 2012. 163(2): p. 154-60.
27. Sonis, S. T., Mucositis: The impact, biology and therapeutic opportunities of oral mucositis. Oral Oncol, 2009. 45(12): p. 1015-20.
28. Sonis, S. T., et al., The gene expression sequence of radiated mucosa in an animal mucositis model. Cell Prolif, 2002. 35 Suppl 1: p. 93-102.
29. Sonis, S. T., et al., Defining mechanisms of action of interleukin-11 on the progression of radiation induced oral mucositis in hamsters. Oral Oncol, 2000. 36(4): p. 373-81.
30. Tezel, A., A. Sens, and S. Mitragotri, Investigations of the role of cavitation in low-frequency sonophoresis using acoustic spectroscopy. J Pharm Sci, 2002. 91(2): p. 444-53.
31. Musther, H., et al., Animal versus human oral drug bioavailability: do they correlate? European journal of pharmaceutical sciences: official journal of the European Federation for Pharmaceutical Sciences, 2014. 57(100): p. 280-291.
32. Ngo, O., et al., Development of low frequency (20-100 kHz) clinically viable ultrasound applicator for chronic wound treatment. IEEE Transactions on Ultrasonics, Ferroelectrics, and Frequency Control, 2018: p. 1-1.
33. Mitragotri, S., D. Blankschtein, and R. Langer, Ultrasound-mediated transdermal protein delivery. Science, 1995. 269(5225): p. 850-3.
34. Paoletti, J. and K. McCord, Compounding mouthwashes and rinses for oral ulcerations. Int J Pharm Compd, 1999. 3(1): p. 8-10.
35. Khojasteh, S. C., H. Wong, and C.E.C.A. Hop, Drug Metabolism and Pharmacokinetics Quick Guide. 2011: Springer New York.
36. Vigarios, E., J. B. Epstein, and V.J.S.C.i.C. Sibaud, Oral mucosal changes induced by anticancer targeted therapies and immune checkpoint inhibitors. 2017. 25(5): p. 1713-1739.
37. Peterson, D. E., R. Srivastava, and R. V. Lalla, Oral mucosal injury in oncology patients: perspectives on maturation of a field. Oral Dis, 2015. 21(2): p. 133-41.
38. Zur, E., Oral mucositis: etiology, and clinical and pharmaceutical management. Int J Pharm Compd, 2012. 16(1): p. 22-33.
39. Saunders, D. P., et al., Systematic review of antimicrobials, mucosal coating agents, anesthetics, and analgesics for the management of oral mucositis in cancer patients. Support Care Cancer, 2013. 21(11): p. 3191-207.
40. Logan, R. M., et al., The role of pro-inflammatory cytokines in cancer treatment-induced alimentary tract mucositis: Pathobiology, animal models and cytotoxic drugs. Cancer Treatment Reviews, 2007. 33(5): p. 448-460.
41. Katayama, S., et al., Effect of polaprezinc on healing of acetic acid-induced stomatitis in hamsters. J Pharm Pharm Sci, 2000. 3(1): p. 114-7.
42. Akintoye, S. O. and M. S. Greenberg, Recurrent aphthous stomatitis. Dent Clin North Am, 2014. 58(2): p. 281-97.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A device for delivering an agent into buccal tissue of a subject, the device comprising:
an ultrasound transducer;
a fluid chamber that is configured to fit within an oral cavity of a subject, wherein the fluid chamber is fluidically sealed from the ultrasound transducer and comprises an opening; and
an ultrasound horn that is operably coupled to the ultrasound transducer and extends into the fluid chamber.

2. The device of claim 1, further comprising: an enclosure that encloses a portion of the fluid chamber.

3. The device of claim 2, wherein the enclosure comprises a membrane that is permeable to gas but impermeable to liquid.

4. The device of claim 2, wherein the enclosure comprises at least one exhaust channel fluidically connected to the fluid chamber.

5. The device of claim 2, wherein the enclosure comprises a port that fluidically connects the fluid chamber to a fluid source.

6. The device of claim 1, further comprising: an illumination source that illuminates the opening of the fluid chamber.

7. The device of claim 1, further comprising: a thermocouple that operably couples the fluid chamber to the ultrasound transducer.

8. The device of claim 1, further comprising: a circuit breaker that is operably coupled to the ultrasound transducer and terminates a signal to the ultrasound transducer in response to a stimulus.

9. The device of claim 8, wherein the stimulus is selected from the group consisting of time, temperature, resistivity, and voltage.

10. The device of claim 1, further comprising: a control unit operably coupled to the ultrasound transducer.

11. The device of claim 10, wherein the control unit comprises one selected from the group consisting of an input mechanism, an output mechanism, a logic board, and an ultrasound driver board.

12. A method for delivering an agent into buccal tissue of a subject, the method comprising:
introducing a chamber comprising a fluid and an agent into an oral cavity of a subject; and
delivering ultrasound energy to the fluid in the chamber at a frequency to produce bubbles within the fluid and cause transient cavitation of the bubbles, thereby propelling the agent in the fluid from the chamber and into the buccal tissue of the subject.

13. The method of claim 12, wherein the ultrasound energy is delivered at a frequency of from about 20 kHz to about 60 kHz.

14. The method of claim 12, wherein the ultrasound energy is delivered in a pulse of from about 10 seconds to about 3 minutes.

15. The method of claim 12, wherein the ultrasound energy results in breakdown of less than about 50% of the agent.

16. The method of claim 12, wherein the agent is selected from the group consisting of a nucleic acid, a peptide, a polypeptide, a protein, an antibody, an organic molecule, and any combination thereof.

17. The method of claim 16, wherein the agent is not encapsulated and is not in a complex with other macromolecules.

18. The method of claim 12, wherein at least 10% of the agent is transferred from the chamber to the buccal tissue.

19. The method of claim 12, wherein the fluid comprises an excipient.

20. The method of claim 19, wherein the excipient is selected from the group consisting of 1,2,4,5 benzenetetracarboxylic acid, 3,3′ thiodipropione acid, 8-arm poly(ethylene glycol), adipic acid, alpha-cyclodextrin, cysteine, didodecyl 3,3′-thiodipropionate, EDTA, fructose, glycerin, mannose, mucin, poloxamer 407, poly(lactide glycolide) acid, poly(vinyl alcohol), polyethoxylated castor oil, saccharin, sodium glycolate, sodium glycocholate, sodium taurodeoxycholate, and sodium thiosulfate.

21. The method of claim 12, further comprising: introducing the agent into the chamber.
